(19) United States
(12) Patent Application Publication
(10) Pub. No.: US 2021/0177987 A1
(43) Pub. Date: Jun. 17, 2021

(54) ANTI-TISSUE FACTOR ANTIBODY-DRUG CONJUGATES AND THEIR USE IN THE TREATMENT OF CANCER

(71) Applicant: Genmab A/S, Copenhagen V (DK)

(72) Inventors: Reshma Abdulla RANGWALA, Princeton, NJ (US); Steen LISBY, Hornbaek (DK)

(21) Appl. No.: 16/760,373

(22) PCT Filed: Nov. 1, 2018
(86) PCT No.: PCT/US2018/058771

§ 371 Date: Apr. 29, 2020

Related U.S. Application Data
(60) Provisional application No. 62/580,877, filed on Nov. 2, 2017.

(51) Int. Cl. A61K 47/68; A61P 35/00; C07K 16/32; A61K 9/00; A61K 45/06; A61K 39/395; A61P 35/04

(52) U.S. Cl. CPC A61K 47/6869; A61P 35/00; A61K 47/6811; C07K 16/32; A61K 2039/505; A61K 45/06; A61K 39/39558; A61P 35/04; C07K 2317/565; A61K 9/0019

(57) ABSTRACT

The invention provides methods and compositions for treating cancer, such as advanced cervical cancer, in a subject, such as by the administration of antibody-drug conjugates that bind to tissue factor (TF). The invention also provides articles of manufacture and compositions comprising said antibody drug-conjugates that bind to TF for use in treating cancer (e.g., advanced cervical cancer).

### CROSS-REFERENCE TO RELATED APPLICATION

This application claims priority to U.S. Provisional application No. 62/580,877 filed on Nov. 2, 2017, the contents of which are incorporated herein by reference in their entirety.

### FIELD OF THE INVENTION

The present invention relates to anti-tissue factor (TF) antibody-drug conjugates and methods of using the same to treat cancer, such as advanced cervical cancer.

### BACKGROUND OF THE INVENTION

Tissue factor (TF), also called thromboplastin, factor III or CD142 is a protein present in subendothelial tissue, platelets, and leukocytes necessary for the initiation of thrombin formation from the zymogen prothrombin. Thrombin formation ultimately leads to the coagulation of blood. TF enables cells to initiate the blood coagulation cascades, and it functions as the high-affinity receptor for the coagulation factor VII (FVII), a serine protease. The resulting complex provides a catalytic event that is responsible for initiation of the coagulation protease cascades by specific limited proteolysis. Unlike the other cofactors of these protease cascades, which circulate as nonfunctional precursors, TF is a potent initiator that is fully functional when expressed on cell surfaces.

TF is the cell surface receptor for the serine protease factor VIIa (FVIIa). Binding of FVIIa to TF starts signaling processes inside the cell, said signaling function playing a role in angiogenesis. Whereas angiogenesis is a normal process in growth and development, as well as in wound healing, it is also a fundamental step in the transition of tumors from a dormant state to a malignant state. When cancer cells gain the ability to produce proteins that participate in angiogenesis (i.e., angiogenic growth factors), these proteins are released by the tumor into nearby tissues, thereby stimulating new blood vessels to sprout from existing healthy blood vessels toward and into the tumor. Once new blood vessels enter the tumor, the tumor can rapidly expand its size and invade local tissue and organs. Through the new blood vessels, cancer cells may further escape into the circulation and lodge in other organs to form new tumors, also known as metastasis.

TF expression is observed in many types of cancer, including cervical cancer, and is associated with more aggressive disease. Furthermore, human TF also exists in a soluble alternatively-spliced form, asHTF. It has recently been found that asHTF promotes tumor growth (Hobbs et al., 2007, Thrombosis Res. 120(2): S13-S21).

Cervical cancer poses a significant medical problem worldwide with an estimated incidence of more than 500,000 new cases and 250,000 deaths annually. See Tewari et al., 2014, N Engl J Med., 370:734-743. In the Europe Union, approximately 34,000 new cases of cervical cancer and 13,000 deaths occur annually. See Hillemanns et al., 2016, Oncol. Res. Treat. 39:501-506. The main types of cervical cancer are squamous cell carcinoma and adenocarcinoma. Long-lasting infections with human papillomavirus (HPV) type 16 and 18 cause most cases of cervical cancer. The standard for first-line therapy of cervical cancer was a platinum-plus a taxane-based therapy. Bevacizumab, an anti-VEGF antibody, was approved by the U.S. Food and Drug Administration for use in combination with chemotherapy for the treatment of cervical cancer, which had improved overall survival in clinical trials. First-line (1L) treatment for advanced cervical cancer is comprised of bevacizumab combined with paclitaxel plus a platinum (e.g., cisplatin or carboplatin) or paclitaxel plus topotecan. Despite a 48% objective response rate (ORR) and a median overall survival (OS) of approximately 18 months, unfortunately almost all patients relapse after this 1L treatment. See Tewari et al., 2014, N Engl J Med., 370:734-743. For second-line (2L) treatment, no approved therapy is available and patients are often treated with single agent modalities including, but not limited to: pemetrexed, topotecan, docetaxel, nab-paclitaxel, vinorelbine and in some cases bevacizumab. A meta-analysis of single agent treatment demonstrates a modest response rate of only 10.9% (i.e., 60 responders out of 552 patients) and median overall survivals (OS) of approximately 7 months. See e.g., Burotto et al., 2015, Oncologist 20:725-726; Candelaria et al., 2009, Int. I Gynecol. Cancer. 19:1632-1637; Coronel et al., 2009, Med. Oncol. 26:210-214; Fiorica et al., 2009, Gynecol. Oncol. 115:285-289; Garcia et. al., 2007, Am. J. Clin. Oncol. 30-428-431; Goncalves et al., 2008, Gynecol. Oncol. 108:42-46; Homesley et al., 2008, Int. J. Clin. Oncol. 13:62-65; McLachlan et al., 2017, Clin. Oncol. (R. Coll. Radiol.) 29:153-160; Miller et al., 2008, Gynecol. Oncol. 110:65-70; Monk et al., 2009, J. Clin. Oncol. 27:1069-1074; Muggia et al., 2004, Gynecol. Oncol. 92:639-643; Rose et al., 2006, Gynecol. Oncol. 102:210-213; Santin et al., 2011, Gynecol. Oncol. 122:495-500; Schilder et al., 2005, Gynecol. Oncol. 96:103-107; and Torfs et al., 2012, Eur. J Cancer. 48:1332-1340. The five year relative survival for stage IV cervical cancer is only 15%, demonstrating a high need for improved methods of treating cervical cancer.

The present invention meets this need by providing highly specific and effective anti-TF antibody-drug conjugates, in particular for the use in the treatment of cervical cancer.

All references cited herein, including patent applications, patent publications, and scientific literature, are herein incorporated by reference in their entirety, as if each individual reference were specifically and individually indicated to be incorporated by reference.

### SUMMARY

Provided herein are methods of treating cervical cancer in a subject comprising administering to the subject an antibody-drug conjugate that binds to tissue factor (TF). In some aspects, provided herein is a method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof (e.g., a functional peptide analog) or a functional derivative thereof, and wherein the antibody-drug conjugate is administered at a dose ranging from about 1.5 mg/kg to about 2.1 mg/kg. In a further embodiment, the dose is about 2.0 mg/kg. In some of any of the embodiments herein, the antibody-drug conjugate is administered once about every 1 week, 2 weeks, 3 weeks or 4 weeks. In some of any of the embodiments herein, the antibody-drug conjugate is administered once about every 3 weeks. In some of any of the embodiments herein, the subject has been previously treated with one or more therapeutic agents and did not respond to the treatment, wherein the one or more therapeutic agents is not the antibody-drug conjugate. In some of any of the embodiments herein, the subject has been previously treated with one or more therapeutic agents and relapsed after the treatment, wherein the one or more therapeutic agents is not the antibody-drug conjugate. In some of any of the embodiments herein, the subject has been previously treated with one or more therapeutic agents and has experienced disease progression during the treatment, wherein the one or more therapeutic agents is not the antibody-drug conjugate. In some of any of the embodiments herein, the one or more therapeutic agents comprises a platinum-based therapeutic agent. In some of any of the embodiments herein, the one or more therapeutic agents is selected from the group consisting of: paclitaxel, cisplatin, carboplatin, topotecan, gemcitabine, fluorouracil, ixabepilone, imatinib mesylate, docetaxel, gefitinib, paclitaxel, pemetrexed, vinorelbine, doxil, cetuximab, pembrolizumab, nivolumab and bevacizumab. In some of any of the embodiments herein, the subject has experienced disease progression during or after treatment with: a) paclitaxel and cisplatin, b) paclitaxel and carboplatin, or c) paclitaxel and topotecan. In some of any of the embodiments herein, the subject has received treatment with bevacizumab. In some of any of the embodiments herein, the subject is ineligible for treatment with bevacizumab. In some of any of the embodiments herein, the subject is not a candidate for curative therapy. In some of any of the embodiments herein, the curative therapy comprises radiotherapy and/or exenterative surgery. In some of any of the embodiments herein, the subject did not respond to treatment with no more than two prior systemic treatment regimens. In some of any of the embodiments herein, the subject relapsed after treatment with no more than two prior systemic treatment regimens. In some of any of the embodiments herein, the cervical cancer is an adenocarcinoma, an adenosquamous carcinoma or a squamous cell carcinoma. In some of any of the embodiments herein, the cervical cancer is an advanced stage cervical cancer, such as a stage 3 or stage 4 cervical cancer, such as metastatic cervical cancer. In some of any of the embodiments herein, the cervical cancer is recurrent cervical cancer. In some of any of the embodiments herein, the monomethyl auristatin is monomethyl auristatin E (MMAE). In some of any of the embodiments herein, the anti-TF antibody or antigen-binding fragment thereof of the antibody-drug conjugate is a monoclonal antibody or a monoclonal antigen-binding fragment thereof. In some of any of the embodiments herein, the anti-TF antibody or antigen-binding fragment thereof of the antibody-drug conjugate comprises a heavy chain variable region and a light chain variable region, wherein the heavy chain variable region comprises:

(i) a CDR-H1 comprising the amino acid sequence of SEQ ID NO:1;

(ii) a CDR-H2 comprising the amino acid sequence of SEQ ID NO:2; and

(iii) a CDR-H3 comprising the amino acid sequence of SEQ ID NO:3; and wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof of the antibody-drug conjugate are defined by the IMGT numbering scheme.

In some of any of the embodiments herein, the anti-TF antibody or antigen-binding fragment thereof of the antibody-drug conjugate comprises a heavy chain variable region comprising an amino acid sequence at least about 85%, at least about 90%, or at least about 95% identical to the amino acid sequence of SEQ ID NO:7 and a light chain variable region comprising an amino acid sequence at least about 85%, at least about 90%, or at least about 95% identical to the amino acid sequence of SEQ ID NO:8. In some of any of the embodiments herein, the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8. In some of any of the embodiments herein, the anti-TF antibody of the antibody-drug conjugate is tisotumab. In some of any of the embodiments herein, the antibody-drug conjugate further comprises a linker between the anti-TF antibody or antigen-binding fragment thereof and the monomethyl auristatin. In a further embodiment, the linker is a cleavable peptide linker. In a further embodiment, the cleavable peptide linker has a formula: -MC-vc-PAB-, wherein:

a) MC is:

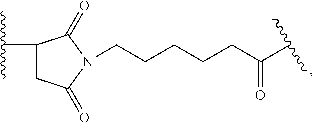 [SMILES: *C(=O)CCCCCN1C(=O)CC(*)C1=O]

b) vc is the dipeptide valine-citrulline, and

c) PAB is:

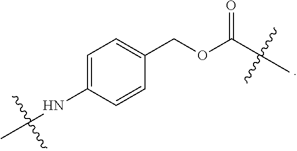 [SMILES: *Nc1ccc(COC(*)=O)cc1]

In some of any of the embodiments herein, the linker is attached to sulphydryl residues of the anti-TF antibody obtained by partial reduction or full reduction of the anti-TF antibody or antigen-binding fragment thereof. In a further embodiment, the linker is attached to MMAE, wherein the antibody-drug conjugate has the following structure:

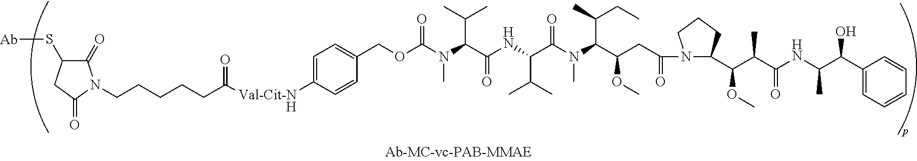

wherein p denotes a number from 1 to 8, S represents a sulphydryl residue of the anti-TF antibody, and Ab designates the anti-TF antibody or antigen-binding fragment thereof. In a further embodiment, the average value of p in a population of the antibody-drug conjugates is about 4. In some of any of the embodiments herein, the antibody-drug conjugate is tisotumab vedotin. In some of any of the embodiments herein, the route of administration for the antibody-drug conjugate is intravenous (e.g., intravenous infusion). In some of any of the embodiments herein, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF. In some of any of the embodiments herein, one or more therapeutic effects in the subject is improved after administration of the antibody-drug conjugate relative to a baseline. In a further embodiment, the one or more therapeutic effects is selected from the group consisting of: size of a tumor derived from the cervical cancer, objective response rate, duration of response, time to response, progression free survival, and overall survival. In some of any of the embodiments herein, the size of a tumor derived from the cervical cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cervical cancer before administration of the antibody-drug conjugate. In some of any of the embodiments herein, the objective response rate is at least about 20%, at least about 25%, at least about 30%, at least about 35%, at least about 40%, at least about 45%, at least about 50%, at least about 60%, at least about 70%, or at least about 80%. In some of any of the embodiments herein, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some of any of the embodiments herein, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some of any of the embodiments herein, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some of any of the embodiments herein, the subject has one or more adverse events and is further administered an additional therapeutic agent to eliminate or reduce the severity of the one or more adverse events. In some of any of the embodiments herein, the subject is at risk of developing one or more adverse events and is further administered an additional therapeutic agent to prevent or reduce the severity of the one or more adverse events. In some of any of the embodiments herein, the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration. In some of any of the embodiments herein, the one or more adverse events is a grade 3 or greater adverse event. In some of any of the embodiments herein, the one or more adverse events is a serious adverse event. In some of any of the embodiments herein, the one or more adverse events is conjunctivitis and/or keratitis and the additional agent is a preservative-free lubricating eye drop, an ocular vasoconstrictor and/or a steroid eye drop. In some of any of the embodiments herein, the antibody-drug conjugate is administered as a monotherapy. In some of any of the embodiments herein, the subject is a human. In some of any of the embodiments herein, the antibody-drug conjugate is in a pharmaceutical composition comprising the antibody-drug conjugate and a pharmaceutical acceptable carrier.

Also provided herein are articles of manufacture comprising an antibody-drug conjugate that binds to TF. In some aspects, provided herein is an article of manufacture comprising: a) a medicament comprising an antibody-drug conjugate, wherein the antibody drug-conjugate comprises an anti-TF antibody or an antigen-binding fragment thereof conjugated to a monomethyl auristatin or a functional analog thereof (e.g., a functional peptide analog) or a functional derivative thereof; and b) a package insert comprising instructions for administration of the medicament comprising the antibody-drug conjugate in a method of treating cervical cancer in a subject according to some of any of the embodiments herein. In a further embodiment, the medicament comprising the antibody-drug conjugate is in a container selected from group consisting of: a vial, a syringe, and an infusion bag. In a further embodiment, the container comprises the antibody-drug conjugate at a dosage amount from about 4 mg to about 500 mg. In a further embodiment, the container comprises the antibody-drug conjugate at a dosage amount from about 20 mg to about 60 mg. In a further embodiment, the container comprises the antibody-drug conjugate at a dosage amount of about 40 mg. In another further embodiment, the container comprises the antibody-drug conjugate at a dosage amount of 40 mg. In another further embodiment, the container comprises the antibody-drug conjugate at a concentration from about 5 mg/mL to about 15 mg/mL. In some of any of the embodiments herein, the medicament comprising the antibody-drug conjugate is a lyophilized powder. In a further embodiment, the lyophilized powder is reconstituted with a suitable diluent resulting in a final concentration from about 5 mg/mL to about 15 mg/mL. In some of any of the embodiments herein, the medicament comprising the antibody-drug conjugate is for administration by intravenous infusion or injection. In a further embodiment, the medicament comprising the antibody-drug conjugate is for administration by intravenous infusion.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram showing the mechanism of action (MOA) of the antibody-drug conjugate tisotumab vedotin.

FIG. 2 is a diagram showing the dose escalation study design for treatment of cancer patients with tisotumab vedotin. q3w indicates a treatment cycle is every three weeks.

FIG. 3 is a graph showing the most common treatment-related adverse effects (AEs) occurring in ≥4 patients overall after treatment with tisotumab vedotin at all doses tested. N=27 indicates 27 patients.

FIGS. 4A and 4B is a graph showing the A) mean plasma tisotumab vedotin concentration and B) mean plasma free MMAE concentration over time during cycle 1 and cycle 2 for all dose cohorts.

FIG. 5 is a graph showing the best percentage change in tumor size from baseline in 27 patients. (i) indicates Patient 1 with cervical cancer and treated with 2.2 mg/kg of tisotumab vedotin. (ii) indicates Patient 2 with cervical cancer and treated with 1.2 mg/kg of tisotumab vedotin. Baseline was defined as the latest available measurement made before the first treatment with tisotumab vedotin.

FIG. 6 is a computed tomography (CT) scan of lung metastasis of Patient 2. This patient had cervical cancer and was treated with 1.2 mg/kg of tisotumab vedotin.

FIG. 7 is a graph showing the most common adverse events (AEs) in the 34 treated cervical cancer patients.

FIG. 8 is a graph showing the best percentage change from baseline in target lesion. ^{a} indicates that two patients were withdrawn prior to CT scan, and thus not represented in the graph. ^{b} indicates PD due to new lesion at same scan. Baseline was defined as the latest available measurement made before the first treatment with tisotumab vedotin.

FIG. 9 is a graph showing the best percentage change from baseline in target lesion. ^{a} indicates a patient that had lymph node disease and persistent non-target lesions for best response of PR. ^{b} indicates a patient that had lymph node disease, persistent non-target lesions, and a new lesion for best response of PD. Baseline was defined as the latest available measurement made before the first treatment with tisotumab vedotin.

FIG. 10 is a graph showing time to and duration of response. ^{a} Response defined as unconfirmed+confirmed response.

FIG. 11 is a diagram showing the Phase II study design for treatment with tisotumab vedotin in patients with previously treated, recurrent or metastatic cancer who have received at least one prior line of systemic therapy. ^{a} indicates tisotumab vedotin 2.0 mg/kg infusion on day 1 of each cycle until disease progression. Each treatment cycle was 3 weeks (Q3W). ^{b} indicates CT or MRI scan every 6 weeks (±7 days) for the first 30 weeks of treatment and every 12 weeks (±7 days) thereafter regardless of treatment delays. indicates optional.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “tissue factor”, “TF”, “CD142”, “tissue factor antigen”, “TF antigen” and “CD142 antigen” are used interchangeably herein, and, unless specified otherwise, include any variants, isoforms and species homologs of human tissue factor which are naturally expressed by cells or are expressed on cells transfected with the tissue factor gene. Tissue factor may be the sequence Genbank accession NP_001984.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. Each light chain typically is comprised of a light chain variable region (abbreviated herein as V_{L} or VL) and a light chain constant region (C_{L} or CL). The light chain constant region typically is comprised of one domain, C_{L}. The V_{H} and V_{L} regions may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). Each V_{H} and V_{L} is typically composed of three CDRs and four FRs, arranged from amino-terminus to carboxy-terminus in the following order: FR1, CDR1, FR2, CDR2, FR3, CDR3, FR4 (see also Chothia and Lesk J. Mot. Biol. 195, 901-917 (1987)). Typically, the numbering of amino acid residues in this region is performed by the method described in Kabat et al., Sequences of Proteins of Immunological Interest, 5th Ed. Public Health Service, National Institutes of Health, Bethesda, Md. (1991) (phrases such as variable domain residue numbering as in Kabat or according to Kabat herein refer to this numbering system for heavy chain variable domains or light chain variable domains). Using this numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of V_{H} CDR2 and inserted residues (for instance residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more clays, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody herein, unless otherwise stated or clearly contradicted by context, includes fragments of an antibody that retain the ability to specifically bind to the antigen (e.g., antigen-binding fragment). It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the V_{L}, V_{H}, C_{L} and C_{H}1 domains, or a monovalent antibody as described in WO2007059782 (Genmab A/S); (ii) F(ab′)₂ fragments, bivalent fragments comprising two Fab fragments linked by a disulfide bridge at the hinge region; (iii) a Fd fragment consisting essentially of the V_{H} and C_{H}1 domains; (iv) a Fv fragment, consisting essentially of the V_{L} and V_{H} domains of a single arm of an antibody, (v) a dAb fragment (Ward et al., Nature 341, 544-546 (1989)), which consists essentially of a V_{H} domain and also called domain antibodies (Holt et al; Trends Biotechnol, 2003 November; 21(11):484-90); (vi) camelid or nanobodies (Revets et al; Expert Opin Biol Ther. 2005 January; 5(1): 111-24) and (vii) an isolated complementarity determining region (CDR). Furthermore, although the two domains of the Fv fragment, V_{L} and V_{H}, are coded for by separate genes, they may be joined, using recombinant methods, by a synthetic linker that enables them to be made as a single protein chain in which the V_{L} and V_{H} regions pair to form monovalent molecules (known as single chain antibodies or single chain Fv (scFv), see for instance Bird et ai., Science 242, 423-426 (1988) and Huston et al., PNAS USA 85, 5879-5883 (1988)). Such single chain antibodies are encompassed within the term antibody unless otherwise noted or clearly indicated by context. Although such antigen-binding fragments are generally included within the meaning of antibody, they collectively and each independently are unique features of the present invention, exhibiting different biological properties and utility. These and other useful antibody fragments in the context of the present invention are discussed further herein. It also should be understood that, the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (e.g., antigen-binding fragments) provided by any known technique, such as enzymatic cleavage, peptide synthesis, and recombinant, techniques. An antibody as generated can possess any isotype. Where not expressly stated, and unless the context indicates otherwise, the term “antibody” also includes an antigen-binding fragment or an antigen-binding portion of any of the aforementioned immunoglobulins.

An “isolated antibody” refers to an antibody that is substantially free of other antibodies having different antigenic specificities (e.g., an isolated antibody that binds specifically to TF is substantially free of antibodies that bind specifically to antigens other than TF). An isolated antibody that binds specifically to TF can, however, have cross-reactivity to other antigens, such as TF molecules from different species. Moreover, an isolated antibody can be substantially free of other cellular material and/or chemicals. In one embodiment, an antibody includes a conjugate attached to another agent (e.g., small molecule drug). In some embodiments, an anti-TF antibody includes a conjugate of an anti-TF antibody with a small molecule drug (e.g., MMAE or MMAF).

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic, or other techniques known to those skilled in the art.

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

A “humanized antibody” refers to an antibody in which some, most, or all of the amino acids outside the CDRs of a non-human antibody are replaced with corresponding amino acids derived from human immunoglobulins. In one embodiment of a humanized form of an antibody, some, most, or all of the amino acids outside the CDRs have been replaced with amino acids from human immunoglobulins, whereas some, most, or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody. In some embodiments, the CDRs of a humanized antibody contain CDRs from a non-human, mammalian antibody. In other embodiments, the CDRs of a humanized antibody contain CDRs from an engineered, synthetic antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-TF antibody binds specifically to TF.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., Sequences of Proteins of Immunological Interest, 5^{th} Ed. Public Health Service, National Institute of Health, Bethesda, Md. (1991)). Chothia HVRs refer instead to the location of the structural loops (Chothia and Lesk J. Mol. Biol. 196:901-917 (1987)). The “contact” HVRs are based on an analysis of the available complex crystal structures. The residues from each of these HVRs are noted below.

| Loop | Kabat | Chothia | Contact |
|---|---|---|---|
| L1 | L24-L34 | L26-L34 | L30-L36 |
| L2 | L50-L56 | L50-L56 | L46-L55 |
| L3 | L89-L97 | L91-L96 | L89-L96 |
| H1 | H31-H35B | H26-H32 | H30-H35B |
| | | | (Kab at Numbering) |
| H1 | H31-H35 | H26-H32 | H30-H35 |
| | | | (Chothia Numbering) |
| H2 | H50-H65 | H53-H56 | H47-H58 |
| H3 | H95-H102 | H95-H102 | H93-H101 |

As used herein, the terms “binding” or “specifically binds” in the context of the binding of an antibody to a pre-determined antigen typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less when determined by for instance surface plasmon resonance (SPR) technology in a BIAcore 3000 Instrument using the antigen as the ligand and the. antibody as the analyte, and binds to the predetermined antigen with an affinity corresponding to a K_{D} that is at least ten-fold lower, such as at least 100 fold lower, for instance at least 1,000 fold lower, such as at. least 10,000 fold lower, for instance at least 100,000 fold lower than its affinity for binding to a non-specific antigen (e.g., BSA, casein) other than the pre-determined antigen or a closely-related antigen. The amount with which the affinity is lower is dependent on the K_{D} of the antibody, so that when the K_{D} of the antibody is very low (that is, the antibody is highly specific), then the amount with which the affinity for the antigen is lower than the affinity for a non-specific antigen may be at least 10,000 fold.

The term “k_{d}” (sec⁻¹), as used herein, refers to the dissociation rate constant of a particular antibody-antigen interaction. Said value is also referred to as the k_{off} value.

The term “kₐ” (M⁻¹×sec⁻¹), as used herein, refers to the association rate constant of a particular antibody-antigen interaction.

The term “K_{D}” (M), as used herein, refers to the dissociation equilibrium constant of a particular antibody-antigen interaction.

The term “K_{A}” (M⁻¹), as used herein, refers to the association equilibrium constant of a particular antibody-antigen interaction and is obtained by dividing the kₐ by the k_{d}.

The term “ADC” refers to an antibody-drug conjugate, which in the context of the present invention refers to an anti-TF antibody, which is coupled to another moiety (e.g., MMAE or MMAF) as described in the present application.

The abbreviations “vc” and “val-cit” refer to the dipeptide valine-citrulline.

The abbreviation “PAB” refers to the self-immolative spacer:

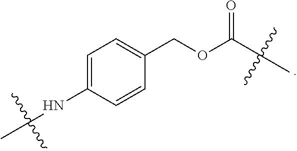 [SMILES: *Nc1ccc(COC(*)=O)cc1]

The abbreviation “MC” refers to the stretcher maleimidocaproyl:

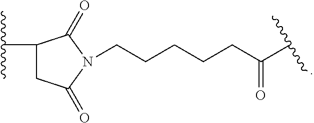 [SMILES: *C(=O)CCCCCN1C(=O)CC(*)C1=O]

The term “Ab-MC-vc-PAB-MMAE” refers to an antibody conjugated to the drug MMAE through a MC-vc-PAB linker.

A “cancer” refers a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a cervical cancer refers to a tumor that is the result of a metastasized cervical cancer.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” refers to an amount effective, at dosages and for periods of time necessary, to achieve a desired therapeutic result. Such desired therapeutic results include protecting a subject against the onset of a disease or promoting disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays. A therapeutically effective amount of an anti-TF antibody-drug conjugate may vary according to factors such as the disease state, age, sex, and weight of the individual, and the ability of the anti-TF antibody-drug conjugate to elicit a desired response in the individual. A therapeutically effective amount is also one in which any toxic or detrimental effects of the anti-TF antibody-drug conjugate are outweighed by the therapeutically beneficial effects.

A therapeutically effective amount of a drug (e.g., anti-TF antibody-drug conjugate) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., an antibody-drug conjugate) that is lower than the usual or typical dose of the therapeutic compound when administered alone for the treatment of a hyperproliferative disease (e.g., cancer).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, at least about 95%, or at least about 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days. Notwithstanding these ultimate measurements of therapeutic effectiveness, evaluation of immunotherapeutic drugs must also make allowance for “immune-related response patterns”.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2. OX, 2.5×, or 3. OX length of the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “ORR”. The sum of complete response (CR) rate and partial response (PR) rate,

As used herein, “overall survival” or “OS” refers to the percentage of individuals in a. group who are likely to be alive after a particular duration of time.

The term “weight-based dose”, as referred to herein, means that a dose administered to a patient is calculated based on the weight of the patient. For example, when a patient with 60 kg body weight requires 2 mg/kg of an anti-TF antibody-drug conjugate, one can calculate and use the appropriate amount of the anti-TF antibody-drug conjugate (i.e., 120 mg) for administration.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., the anti-TF antibody-drug conjugate). For example, a 60 kg person and a 100 kg person would receive the same dose of an antibody-drug conjugate (e.g., 240 mg of an anti-TF antibody-drug conjugate).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” refers to the physical introduction of a therapeutic agent to a subject, using any of the various methods and delivery systems known to those skilled in the art. Exemplary routes of administration for the anti-TF antibody-drug conjugate include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “baseline” or “baseline value” used interchangeably herein can refer to a measurement or characterization of a symptom before the administration of the therapy (e.g., an antibody-drug conjugate as described herein) or at the beginning of administration of the therapy. The baseline value can be compared to a reference value in order to determine the reduction or improvement of a symptom of a TF-associated disease contemplated herein (e.g., cervical cancer). The terms “reference” or “reference value” used interchangeably herein can refer to a measurement or characterization of a symptom after administration of the therapy (e.g., an antibody-drug conjugate as described herein). The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the antibody drug conjugate is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically important”
Requires inpatient hospitalization or prolongation of existing hospitalization, excluding the following: 1) routine treatment or monitoring of the underlying disease, not associated with any deterioration in condition, 2) elective or pre-planned treatment for a pre-existing condition that is unrelated to the indication under study and has not worsened since signing the informed consent, and social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” “once about every three weeks” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Antibody-Drug Conjugates

The present invention provides for anti-TF antibody-drug conjugates that are useful for the treatment of cancer in a subject. In some embodiments, the cancer is cervical cancer. In some embodiments, the cervical cancer is an advanced stage cervical cancer (e.g., stage 3 cervical cancer or stage 4 cervical cancer or metastatic cervical cancer). In some embodiments, the advanced cervical cancer is a metastatic cancer. In some embodiments, the subject has relapsed, recurrent and/or metastatic cervical cancer.

A. Anti-TF Antibody

Generally, antibodies of the disclosure immunospecifically bind TF and exert cytostatic and cytotoxic effects on malignant cells, such as cervical cancer cells. Antibodies of the disclosure are preferably monoclonal, and may be multispecific, human, humanized or chimeric antibodies, single chain antibodies, Fab fragments, F(ab′) fragments, fragments produced by a Fab expression library, and TF binding fragments of any of the above. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the antibodies are human antigen-binding fragments as described herein and include, but are not limited to, Fab, Fab′ and F(ab)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. Preferably, the antibodies or antigen-binding fragments thereof are human, murine (e.g., mouse and rat), donkey, sheep, rabbit, goat, guinea pig, camelid, horse, or chicken.

The antibodies of the present disclosure may be monospecific, bispecific, trispecific or of greater multi specificity. Multispecific antibodies may be specific for different epitopes of TF or may be specific for both TF as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

CDR sequences of the anti-TF antibodies of the anti-TF antibody-drug conjugates provided herein are according to the IMGT numbering scheme as described in Lefranc, M. P. et al., Dev. Comp. Immunol., 2003, 27, 55-77.

In certain embodiments antibodies of the disclosure comprise one or more CDRs of the antibody 011. See WO 2011/157741 and WO 2010/066803. The disclosure encompasses an antibody or derivative thereof comprising a heavy or light chain variable domain, said variable domain comprising (a) a set of three CDRs, in which said set of CDRs are from monoclonal antibody 011, and (b) a set of four framework regions, in which said set of framework regions differs from the set of framework regions in monoclonal antibody 011, and in which said antibody or derivative thereof immunospecifically binds TF. In certain embodiments, the anti-TF antibody is 011. The antibody 011 is also known as tisotumab.

In one aspect, anti-TF antibodies that compete with tisotumab binding to TF are provided. Anti-TF antibodies that bind to the same epitope as tisotumab are also provided.

In one aspect, provided herein is an anti-TF antibody comprising 1, 2, 3, 4, 5, or 6 of the CDR sequences of tisotumab.

In one aspect, provided herein is an anti-TF antibody comprising a heavy chain variable region and a light chain variable region, wherein the heavy chain variable region comprises (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6, wherein the CDRs of the anti-TF antibody are defined by the IMGT numbering scheme.

An anti-TF antibody described herein may comprise any suitable framework variable domain sequence, provided that the antibody retains the ability to bind TF (e.g., human TF). As used herein, heavy chain framework regions are designated “HC-FR1-FR4,” and light chain framework regions are designated “LC-FR1-FR4.” In some embodiments, the anti-TF antibody comprises a heavy chain variable domain framework sequence of SEQ ID NO:9, 10, 11, and 12 (HC-FR1, HC-FR2, HC-FR3, and HC-FR4, respectively). In some embodiments, the anti-TF antibody comprises a light chain variable domain framework sequence of SEQ ID NO:13, 14, 15, and 16 (LC-FR1, LC-FR2, LC-FR3, and LC-FR4, respectively).

In one embodiment, an anti-TF antibody comprises a heavy chain variable domain comprising a framework sequence and hypervariable regions, wherein the framework sequence comprises the HC-FR1-HC-FR4 amino acid sequences of SEQ ID NO:9 (HC-FR1), SEQ ID NO:10 (HC-FR2), SEQ ID NO:11 (HC-FR3), and SEQ ID NO:12 (HC-FR4), respectively; the CDR-H1 comprises the amino acid sequence of SEQ ID NO:1; the CDR-H2 comprises the amino acid sequence of SEQ ID NO:2; and the CDR-H3 comprises the amino acid sequence of SEQ ID NO:3.

In one embodiment, an anti-TF antibody comprises a light chain variable domain comprising a framework sequence and hypervariable regions, wherein the framework sequence comprises the LC-FR1-LC-FR4 amino acid sequences of SEQ ID NO:13 (LC-FR1), SEQ ID NO:14 (LC-FR2), SEQ ID NO:15 (LC-FR3), and SEQ ID NO:16 (LC-FR4), respectively; the CDR-L1 comprises the amino acid sequence of SEQ ID NO:4; the CDR-L2 comprises the amino acid sequence of SEQ ID NO:5; and the CDR-L3 comprises the amino acid sequence of SEQ ID NO:6.

In some embodiments of the anti-TF antibodies described herein, the heavy chain variable domain comprises the amino acid sequence of

(SEQ ID NO: 7)
EVQLLESGGGLVQPGGSLRLSCAASGFTFSNYAMSWVRQAP
GKGLEWVSSISGSGDYTYYTDSVKGRFTISRDNSKNTLYLQ
MNSLRAEDTAVYYCARSPWGYYLDSWGQGTLVTVSS
and
the light chain variable domain comprises
the amino acid sequence of
(SEQ ID NO: 8)
DIQMTQSPPSLSASAGDRVTITCRASQGISSRLAWYQQKPE
KAPKSLIYAASSLQSGVPSRFSGSGSGTDFTLTISSLQPED
FATYYCQQYNSYPYTFGQGTKLEIK.

In some embodiments of the anti-TF antibodies described herein, the heavy chain CDR sequences comprise the following:

a) CDR-H1
(GFTFSNYA (SEQ ID NO: 1));
b) CDR-H2
(ISGSGDYT (SEQ ID NO: 2));
and
c) CDR-H3
(ARSPWGYYLDS (SEQ ID NO: 3)).

In some embodiments of the anti-TF antibodies described herein, the heavy chain FR sequences comprise the following:

a) HC-FR1
(EVQLLESGGGLVQPGGSLRLSCAAS(SEQ ID NO: 9));
b) HC-FR2
(MSWVRQAPGKGLEWVSS (SEQ ID NO: 10));
c) HC-FR3
(YYTDSVKGRFTISRDNSKNTLYL
QMNSLRAEDTAVYYC(SEQ ID NO: 11));
and
d) HC-FR4
(WGQGTLVTVSS(SEQ ID NO: 12)).

In some embodiments of the anti-TF antibodies described herein, the light chain CDR sequences comprise the following:

a) CDR-L1
(QGISSR (SEQ ID NO: 4));
b) CDR-L2
(AAS (SEQ ID NO: 5));
and
c) CDR-L3
(QQYNSYPYT (SEQ ID NO: 6)).

In some embodiments of the anti-TF antibodies described herein, the light chain FR sequences comprise the following:

a) LC-FR1
(DIQMTQSPPSLSASAGDRVTITCRAS (SEQ ID NO: 13));
b) LC-FR2
(LAWYQQKPEKAPKSLIY(SEQ ID NO: 14));
c) LC-FR3
(SLQSGVPSRFSGSGSGTDFTLT
ISSLQPEDFATYYC(SEQ ID NO: 15));
and
d) LC-FR4
(FGQGTKLEIK(SEQ ID NO: 16)).

In some embodiments, provided herein is an anti-TF antibody that binds to TF (e.g., human TF), wherein the antibody comprises a heavy chain variable region and a light chain variable region, wherein the antibody comprises:

(a) heavy chain variable domain comprising:
(1) an HC-FR1 comprising the amino acid sequence of SEQ ID NO:9;
(2) an CDR-H1 comprising the amino acid sequence of SEQ ID NO:1;
(3) an HC-FR2 comprising the amino acid sequence of SEQ ID NO:10;
(4) an CDR-H2 comprising the amino acid sequence of SEQ ID NO:2;
(5) an HC-FR3 comprising the amino acid sequence of SEQ ID NO:11;
(6) an CDR-H3 comprising the amino acid sequence of SEQ ID NO:3; and
(7) an HC-FR4 comprising the amino acid sequence of SEQ ID NO:12, and/or

(b) a light chain variable domain comprising:
(1) an LC-FR1 comprising the amino acid sequence of SEQ ID NO:13;
(2) an CDR-L1 comprising the amino acid sequence of SEQ ID NO:4;
(3) an LC-FR2 comprising the amino acid sequence of SEQ ID NO:14;
(4) an CDR-L2 comprising the amino acid sequence of SEQ ID NO:5;
(5) an LC-FR3 comprising the amino acid sequence of SEQ ID NO:15;
(6) an CDR-L3 comprising the amino acid sequence of SEQ ID NO:6; and
(7) an LC-FR4 comprising the amino acid sequence of SEQ ID NO:16.

In one aspect, provided herein is an anti-TF antibody comprising a heavy chain variable domain comprising the amino acid sequence of SEQ ID NO:7 or comprising a light chain variable domain comprising the amino acid sequence of SEQ ID NO:8. In one aspect, provided herein is an anti-TF antibody comprising a heavy chain variable domain comprising the amino acid sequence of SEQ ID NO:7 and comprising a light chain variable domain comprising the amino acid sequence of SEQ ID NO:8.

In some embodiments, provided herein is an anti-TF antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDR s (i.e., in the FRs). In some embodiments, the anti-TF antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3.

In some embodiments, provided herein is an anti-TF antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8 contains substitutions (e.g., conservative substitutions), insertions, or deletions relative to the reference sequence and retains the ability to bind to a TF (e.g., human TF). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDR s (i.e., in the FRs). In some embodiments, the anti-TF antibody comprises a light chain variable domain sequence of SEQ ID NO:8 including post-translational modifications of that sequence. In a particular embodiment, the light chain variable domain comprises one, two or three CDRs selected from: (a) CDR-L1 comprising the amino acid sequence of SEQ ID NO:4, (b) CDR-L2 comprising the amino acid sequence of SEQ ID NO:5, and (c) CDR-L3 comprising the amino acid sequence of SEQ ID NO:6.

In some embodiments, the anti-TF antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-TF antibody of the anti-TF antibody-drug conjugate comprises: i) a heavy chain CDR1 set out in SEQ ID NO: 1, a heavy chain CDR2 set out in SEQ ID NO: 2, a heavy chain CDR3 set out in SEQ ID NO: 3; and ii) a light chain CDR1 set out in SEQ ID NO: 4, a light chain CDR2 set out in SEQ ID NO: 5, and a light chain CDR3 set out in SEQ ID NO: 6, wherein the CDRs of the anti-TF antibody of the antibody-drug conjugate are defined by the IMGT numbering scheme.

In some embodiments, the anti-TF antibody of the anti-TF antibody-drug conjugate comprises: i) an amino acid sequence at least 85% identical to a heavy chain variable region set out in SEQ ID NO: 7, and ii) an amino acid sequence at least 85% identical to a light chain variable region set out in SEQ ID NO: 8.

In some embodiments, the anti-TF antibody of the anti-TF antibody-drug conjugate is a monoclonal antibody.

In some embodiments, the anti-TF antibody of the anti-TF antibody-drug conjugate is tisotumab, which is also known as antibody 011 as described in WO 2011/157741 and WO 2010/066803.

Antibodies of the present invention may also be described or specified in terms of their binding affinity to TF. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶M, 5×10⁻⁷ M, 10⁻⁷ M, 5×10⁻⁸ M, 10⁻⁸M, 5×10⁻⁹M, 10⁻⁹ M, 5×10⁻¹⁰ M, 10⁻¹⁰ M, 5×10⁻¹¹ M, 10⁻¹¹M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to TF or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

B. Antibody-Drug Conjugate Structure

In some aspects, the anti-TF antibody-drug conjugates described herein comprise a linker between an anti-TF antibody or antigen-binding fragment thereof as described herein and a cytostatic or cytotoxic drug. In some embodiments the linker is a non-cleavable linker. In some embodiments the linker is a cleavable linker.

In some embodiments, the linker is a cleavable peptide linker comprising maleimido caproyl (MC), the dipeptide valine-citrulline (vc) and p-aminobenzylcarbamate (PAB). In some embodiments, the cleavable peptide linker has the formula: MC-vc-PAB-, wherein:

a) MC is:

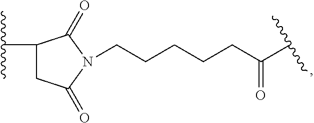 [SMILES: *C(=O)CCCCCN1C(=O)CC(*)C1=O]

b) vc is the dipeptide valine-citrulline, and

c) PAB is:

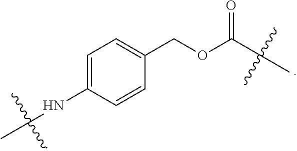 [SMILES: *Nc1ccc(COC(*)=O)cc1]

In some embodiments, the linker is a cleavable peptide linker comprising maleimido caproyl (MC). In some embodiments, the cleavable peptide linker has the formula: MC-, wherein:

a) MC is:

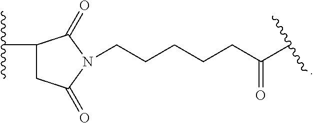 [SMILES: *C(=O)CCCCCN1C(=O)CC(*)C1=O]

In some embodiments, the linker is attached to sulphydryl residues of the anti-TF antibody or antigen-binding fragment thereof obtained by partial or full reduction of the anti-TF antibody or antigen-binding fragment thereof. In some embodiments, the linker is attached to sulphydryl residues of the anti-TF antibody or antigen-binding fragment thereof obtained by partial reduction of the anti-TF antibody or antigen-binding fragment thereof. In some embodiments, the linker is attached to sulphydryl residues of the anti-TF antibody or antigen-binding fragment thereof obtained by full reduction of the anti-TF antibody or antigen-binding fragment thereof.

In some aspects, the anti-TF antibody-drug conjugates described herein comprise a linker as described herein between an anti-TF antibody or antigen-binding fragment thereof as described herein and a cytostatic or cytotoxic drug. Auristatins have been shown to interfere with microtubule dynamics, GTP hydrolysis and nuclear and cellular division (See Woyke et al (2001) Antimicrob. Agents and Chemother. 45(12): 3580-3584) and have anti-cancer (See U.S. Pat. No. 5,663,149) and antifungal activity (See Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023. In some embodiments of the anti-TF antibody-drug conjugates described herein, the cytostatic or cytotoxic drug is an auristatin or a functional analog thereof (e.g., functional peptide thereof) or a functional derivative thereof. In some embodiments, the auristatin is a monomethyl auristatin or a functional analog thereof (e.g., functional peptide thereof) or a functional derivative thereof.

In one embodiment, the auristatin is monomethyl auristatin E (MMAE):

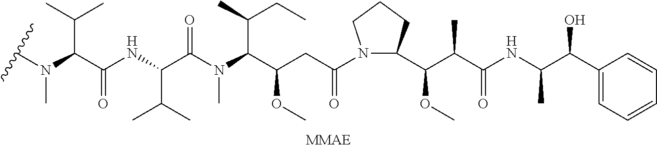 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the attachment site for the linker.

In one embodiment, the auristatin is monomethyl auristatin F (MMAF):

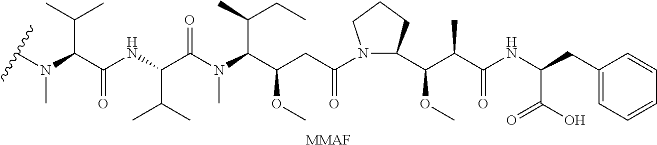

wherein the wavy line indicates the attachment site for the linker.

In one embodiment, the cleavable peptide linker has the formula: MC-vc-PAB-, and is attached to MMAE. The resulting linker-auristatin, MC-vc-PAB-MMAE is also designated vcMMAE. The vcMMAE drug linker moiety and conjugation methods are disclosed in WO2004010957, U.S. Pat. Nos. 7,659,241, 7,829,531 and 7,851,437. When vcMMAE is attached to an anti-TF antibody or antigen-binding fragment thereof as described herein, the resulting structure is:

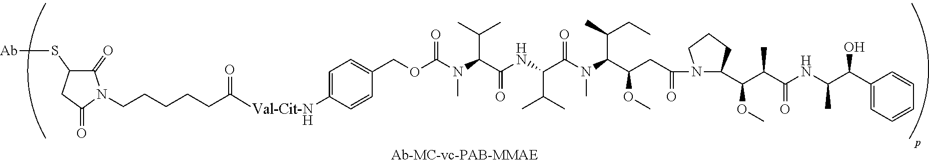

wherein p denotes a number from 1 to 8, e.g., p may be from 3-5, S represents a sulphydryl residue of the anti-TF antibody and Ab designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the cleavable peptide linker has the formula: MC-vc-PAB-, and is attached to MMAF. The resulting linker-auristatin, MC-vc-PAB-MMAF is also designated vcMMAF. In another embodiment, a non-cleavable linker MC is attached to MMAF. The resulting linker-auristatin MC-MMAF is also designated mcMMAF. The vcMMAF and mcMMAF drug linker moieties and conjugation methods are disclosed in WO2005081711 and U.S. Pat. No. 7,498,298. When vcMMAF or mcMMAF is attached to an anti-TF antibody or antigen-binding fragment thereof as described herein, the resulting structure is:

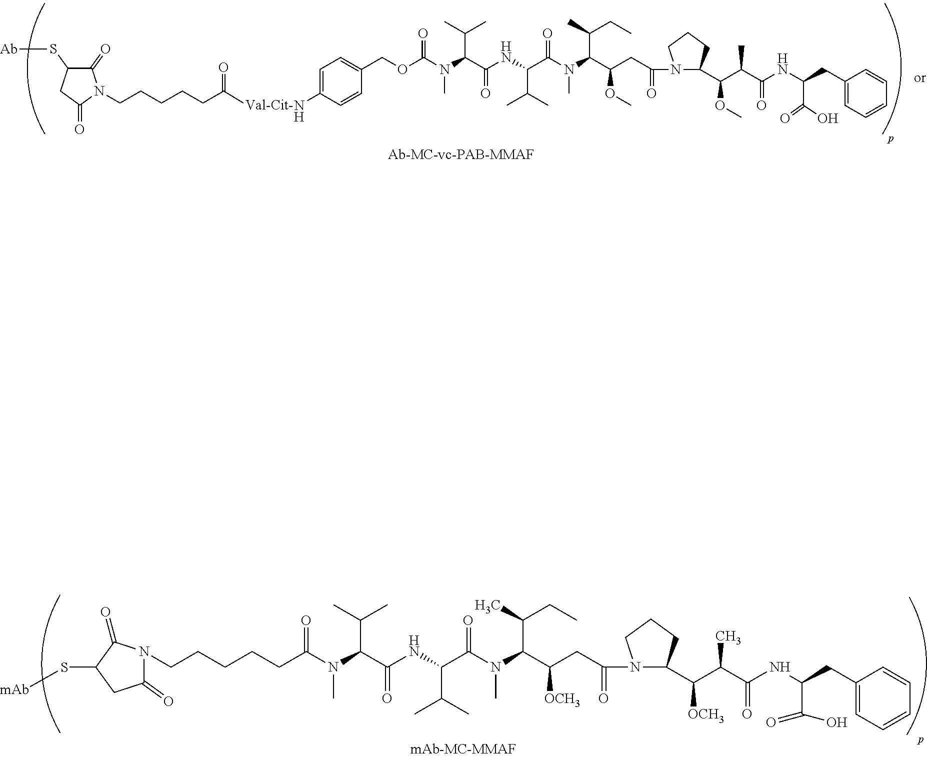

wherein p denotes a number from 1 to 8, e.g., p may be from 3-5, S represents a sulphydryl residue of the anti-TF antibody and Ab or mAb designates an anti-TF antibody or antigen-binding fragment thereof as described herein. In one embodiment, the average value of p in a population of antibody-drug conjugates is about 4. In some embodiments, p is measured by hydrophobic interaction chromatography (HIC), for example by resolving drug-loaded species based on the increasing hydrophobicity with the least hydrophobic, unconjugated form eluting first and the most hydrophobic, 8-drug form eluting last with the area percentage of a peak representing the relative distribution of the particular drug-loaded antibody-drug conjugate species. See Ouyang, J., 2013, Antibody-Drug Conjugates, Methods in Molecular Biology (Methods and Protocols). In some embodiments, p is measured by reversed phase high-performance liquid chromatography (RP-HPLC), for example by first performing a reduction reaction to completely dissociate the heavy and light chains of the ADC, then separating the light and heavy chains and their corresponding drug-loaded forms on an RP column, where the percentage peak are from integration of the light chain and heavy chain peaks, combined with the assigned drug load for each peak, is used to calculate the weighted average drug to antibody ration. See Ouyang, J., 2013, Antibody-Drug Conjugates, Methods in Molecular Biology (Methods and Protocols).

In one embodiment, the antibody-drug conjugate is tisotumab vedotin.

C. Nucleic Acids, Host Cells and Methods of Production

In some aspects, also provided herein are nucleic acids encoding an anti-TF antibody or antigen-binding fragment thereof as described herein. Further provided herein are vectors comprising the nucleic acids encoding an anti-TF antibody or antigen-binding fragment thereof as described herein. Further provided herein are host cells expressing the nucleic acids encoding an anti-TF antibody or antigen-binding fragment thereof as described herein. Further provided herein are host cells comprising the vectors comprising the nucleic acids encoding an anti-TF antibody or antigen-binding fragment thereof as described herein. Methods of producing the anti-TF antibody, linker and antibody-drug conjugate are described in U.S. Pat. No. 9,168,314.

The anti-TF antibodies described herein may be prepared by well-known recombinant techniques using well known expression vector systems and host cells. In one embodiment, the antibodies are prepared in a CHO cell using the GS expression vector system as disclosed in De la Cruz Edmunds et al., 2006, Molecular Biotechnology 34; 179-190, EP216846, U.S. Pat. No. 5,981,216, WO 87/04462, EP323997, U.S. Pat. Nos. 5,591,639, 5,658,759, EP338841, U.S. Pat. Nos. 5,879,936, and 5,891,693.

After isolating and purifying the antibodies from the cell media using well known techniques in the art, they are conjugated with an auristatin via a linker as described in U.S. Pat. No. 9,168,314.

Monoclonal anti-TF antibodies described herein may e.g. be produced by the hybridoma method first described by Kohler et al., Nature, 256, 495 (1975), or may be produced by recombinant DNA methods. Monoclonal antibodies may also be isolated from phage antibody libraries using the techniques described in, for example, Clackson et al., Nature, 352, 624-628 (1991) and Marks et al., J Mol, Biol., 222(3):581-597 (1991). Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc.

In one embodiment, the antibody of the invention is a human antibody. Human monoclonal antibodies directed against tissue factor may be generated using transgenic or transchromosomal mice carrying parts of the human immune system rather than the mouse system. Such transgenic and transchromosomic mice include mice referred to herein as HuMAb mice and KM mice, respectively, and are collectively referred to herein as “transgenic mice”.

The HuMAb mouse contains a human immunoglobulin gene minilocus that encodes unrearranged human heavy (μ and γ) and κ light chain immunoglobulin sequences, together with targeted mutations that inactivate the endogenous μ and κ chain loci (Lonberg, N. et al., Nature, 368, 856-859 (1994)). Accordingly, the mice exhibit reduced expression of mouse IgM or κ and in response to immunization, the introduced human heavy and light chain transgenes undergo class switching and somatic mutation to generate high affinity human IgG, κ monoclonal antibodies (Lonberg, N. et al. (1994), supra; reviewed in Lonberg, N. Handbook of Experimental Pharmacology 113, 49-101 (1994), Lonberg, N. and Huszar. D., Intern. Rev. Immunol, Vol. 13 65-93 (1995) and Harding, F. and Lonberg, N. Ann, N.Y. Acad. Sci 764:536-546 (1995)). The preparation of HuMAb mice is described in detail in Taylor, L. et al., Nucleic Acids Research. 20:6287-6295 (1992), Chen, J. et al., International Immunology. 5:647-656 (1993), Tuaillon at al., J. Immunol, 152:2912-2920 (1994), Taylor, L. et al., International Immunology, 6:579-591 (1994), Fishwild, D. et al., Nature Biotechnology, 14:845-851 (1996). See also U.S. Pat. Nos. 5,545,806, 5,569,825, 5,625,126, 5,633,425, 5,789,650, 5,877,397, 5,661,016, 5,814,318, 5,874,299, 5,770,429, 5,545,807, WO 98/24884, WO 94/25585, WO 93/1227, WO 92/22645, WO 92/03918 and WO 01/09187.

The HCo7 mice have a JKD disruption in their endogenous light chain (kappa) genes (as described in Chen et al, EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo7 human heavy chain transgene (as described in U.S. Pat. No. 5,770,429).

The HCo12 mice have a JKD disruption in their endogenous light chain (kappa) genes (as described in Chen et al., EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo12 human heavy chain transgene (as described in Example 2 of WO 01/14424).

The HCo17 transgenic mouse strain (see also US 2010/0077497) was generated by coinjection of the 80 kb insert of pHC2 (Taylor et al. (1994) Int. Immunol., 6:579-591), the Kb insert of pVX6, and a −460 kb yeast artificial chromosome fragment of the yIgH24 chromosome. This line was designated (HCo17) 25950. The (HCo17) 25950 line was then bred with mice comprising the CMD mutation (described in Example 1 of PCT Publication WO 01109187), the JKD mutation (Chen et al, (1993) EMBO J. 12:811-820), and the (KC05) 9272 transgene (Fishwild et al. (1996) Nature Biotechnology, 14:845-851). The resulting mice express human immunoglobulin heavy and kappa light chain trans genes in a background homozygous for disruption of the endogenous mouse heavy and kappa light chain loci.

The HCo20 transgenic mouse strain is the result of a co-injection of minilocus 30 heavy chain transgene pHC2, the germline variable region (Vh)-containing YAC yIgH10, and the minilocus construct pVx6 (described in WO09097006). The (HCo20) line was then bred with mice comprising the CMD mutation (described in Example 1 of PCT Publication WO 01/09187), the JKD mutation (Chen et al. (1993) EMBO J. 12:811-820), and the (KCO5) 9272 trans gene (Fishwild eta). (1996) Nature Biotechnology, 14:845-851). The resulting mice express human 10 immunoglobulin heavy and kappa light chain transgenes in a background homozygous for disruption of the endogenous mouse heavy and kappa light chain loci.

In order to generate HuMab mice with the salutary effects of the Balb/c strain, HuMab mice were crossed with KCO05 [MIK] (Balb) mice which were generated by backcrossing the KC05 strain (as described in Fishwild et (1996) Nature Biotechnology, 14:845-851) to wild-type Balb/c mice to generate mice as described in WO09097006. Using this crossing Balb/c hybrids were created for HCo12, HCo17, and HCo20 strains.

In the KM mouse strain, the endogenous mouse kappa light chain gene has been homozygously disrupted as described in Chen et al., EMBO J. 12:811-820 (1993) and the endogenous mouse heavy chain gene has been homozygously disrupted as described in Example 1 of WO 01/09187, This mouse strain carries a human kappa light chain transgene, KCo5, as described in Fishwild et al., Nature Biotechnology, 14:845-851 (1996). This mouse strain also carries a human heavy chain transchromosome composed of chromosome 14 fragment hCF (SC20) as described in WO 02/43478.

Splenocytes from these transgenic mice may be used to generate hybridomas that secrete human monoclonal antibodies according to well-known techniques, Human monoclonal or polyclonal antibodies of the present invention, or antibodies of the present invention originating from other species may also be generated transgenically through the generation of another non-human mammal or plant that is transgenic for the immunoglobulin heavy and light chain sequences of interest and production of the antibody in a recoverable form therefrom. In connection with the transgenic production in mammals, antibodies may be produced in, and recovered from, the milk of goats, cows, or other mammals. See for instance U.S. Pat. Nos. 5,827,690, 5,756,687, 5,750,172 and 5,741,957.

Further, human antibodies of the present invention or antibodies of the present invention from other species may be generated through display-type technologies, including, without limitation, phage display, retroviral display, ribosomal display, and other techniques, using techniques well known in the art and the resulting molecules may be subjected to additional maturation, such as affinity maturation, as such techniques are well known in the art (See for instance Hoogenboom et al., J. Mol, Biol. 227(2):381-388 (1992) (phage display), Vaughan et al., Nature Biotech, 14:309 (1996) (phage display), Hanes and Plucthau, PNAS USA 94:4937-4942 (1997) (ribosomal display), Parmley and Smith, Gene, 73:305-318 (1988) (phage display), Scott, TIBS. 17:241-245 (1992), Cwirla et al., PNAS USA, 87:6378-6382 (1990), Russel et al., Nucl. Acids Research, 21:1081-4085 (1993), Hogenboom et al., Immunol, Reviews, 130:43-68 (1992), Chiswell and McCafferty, TIBTECH, 10:80-84 (1992), and U.S. Pat. No. 5,733,743). If display technologies are utilized to produce antibodies that are not human, such antibodies may be humanized.

### III. Methods of Treatment

A. Cervical Cancer

Cervical cancer remains to be one of the leading causes of cancer-related death in women despite advances in screening, diagnosis, prevention, and treatment. It accounts for ˜4% of the total newly diagnosed cancer cases and 4% of the total cancer deaths. See Zhu et al., 2016, Drug Des. Devel. Ther. 10:1885-1895. Cervical cancer is the 7^{th} most common female cancer worldwide and the 16^{th} most common cancer in the European Union. Depending on the stage at initial presentation, cervical cancer will recur in 25-61% of women. See Tempfer et al., 2016, Oncol. Res. Treat. 39:525-533. In most cases, recurrent disease is diagnosed within 2 years of the initial treatment and may be observed in various sites. Chemotherapy is the standard treatment for these patients. See Zhu et al., 2016, Drug Des. Devel. Ther. 10:1885-1895. The median overall survival exceeds one year now, however, the five year relative survival for stage IV cervical cancer is only 15%, demonstrating the high need for improved methods of treating cervical cancer.

The invention provides methods for treating cervical cancer with an antibody-drug conjugate described herein. In a preferred aspect, the antibody-drug conjugate is tisotumab vedotin. In one aspect, the antibody-drug conjugates described herein are for use in a method of treating cervical cancer in a subject. In some embodiments, the subject has not previously received treatment for the cervical cancer. In some embodiments, the subject has received at least one previous treatment for the cervical cancer. In some embodiments, the subject was previously treated with bevacizumab. In some embodiments, the subject is ineligible for treatment with bevacizumab. In some embodiments, the subject is not a candidate for curative therapy. In some embodiments, the curative therapy is radiotherapy and/or exenterative therapy. In some embodiments, the curative therapy is radiotherapy. In some embodiments, the curative therapy is exenterative therapy. In a particular embodiment, the subject is a human.

In some embodiments of the methods or uses provided herein, the cervical cancer is an adenocarcinoma, an adenosquamous carcinoma, a squamous cell carcinoma, a small cell carcinoma, a neuroendocrine tumor, a glassy cell carcinoma or a villoglandular adenocarcinoma. In some embodiments, the cervical cancer is an adenocarcinoma, an adenosquamous carcinoma or a squamous cell carcinoma. In some embodiments, the cervical cancer is an adenocarcinoma. In some embodiments, the cervical cancer is an adenosquamous carcinoma. In some embodiments, the cervical cancer is a squamous cell carcinoma. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF. In some embodiments, the percentage of cells that express TF is determined using immunohistochemistry (IHC). In some embodiments, the percentage of cells that express TF is determined using flow cytometry. In some embodiments, the percentage of cells that express TF is determined using an enzyme-linked immunosorbent assay (ELISA).

In some embodiments of the methods or uses provided herein, the cervical cancer is a stage 0, 1, 2, 3, or 4 cervical cancer. In some embodiments, the cervical cancer is a stage 0, 1A, 1B, 2A, 2B, 3A, 3B, 4A or 4B cervical cancer. In some embodiments, the cervical cancer is staged by the International Federation of Gynecology and Obstetrics (FIGO) staging system. In some embodiments, the staging is based on clinical examination. In some embodiments, in stage 0 cervical cancer the carcinoma is confined to the surface layer (cells lining) the cervix. In some embodiments, in stage 1 cervical cancer the carcinoma has grown deeper into the cervix but has not yet spread beyond it. In some embodiments, in stage 1A cervical cancer the invasive carcinoma can be diagnosed only by microscopy and the deepest invasion is less than 5 mm and the largest extension is less than 7 mm. In some embodiments, in stage 1B cervical cancer the lesions are clinically visible and are limited to the cervix uteri. In some embodiments, in stage 2 cervical cancer the cervical carcinoma has invaded beyond the uterus, but not to the pelvic wall or to the lower third of the vagina. In some embodiments, in stage 2A cervical cancer there is no parametrial invasion. In some embodiments, in stage 2B cervical cancer there is parametrial invasion. In some embodiments, in stage 3 cervical cancer the tumor extends to the pelvic wall and/or involves the lower third of the vagina and/or causes hydronephrosis or non-functioning kidney. In some embodiments, in stage 3A cervical cancer the tumor involves the lower third of the vagina, with no extension to the pelvic wall. In some embodiments, in stage 3B cervical cancer extends to the pelvic wall and/or cause hydronephrosis or non-functioning kidney. In some embodiments, in stage 4 cervical cancer, the carcinoma has extended beyond the true pelvis or has involved the mucosa of the bladder or rectum. In some embodiments, in stage 4A cervical cancer the tumor has spread to adjacent organs. In some embodiments, in stage 4B cervical cancer the tumor has spread to distant organs. In some embodiments, the cervical cancer is an advanced cervical cancer such as a grade 3 or grade 4 cervical cancer. In some embodiments, the advanced cervical cancer is metastatic cervical cancer. In some embodiments, the cervical cancer is metastatic cervical cancer and recurrent cervical cancer. In some embodiments, the cervical cancer is metastatic cervical cancer. In some embodiments, the cervical cancer is recurrent cervical cancer.

In some embodiments of the methods or uses provided herein, the subject has been previously treated for the cervical cancer. In some embodiments, the subject did not respond to the treatment (e.g., the subject experienced disease progression during treatment). In some embodiments, the one or more therapeutic agents administered to the subject was not an anti-TF antibody-drug conjugate as described herein. In some embodiments, the one or more therapeutic agents administered to the subject was paclitaxel, cisplatin, carboplatin, topotecan, gemcitabine, fluorouracil, ixabepilone, imatinib mesylate, docetaxel, gefitinib, paclitaxel, pemetrexed, vinorelbine, doxil, cetuximab, pembrolizumab, nivolumab, bevacizumab, or any combination thereof. In some embodiments, the one or more therapeutic agents administered to the subject was a platinum-based therapeutic agent. In some embodiments, the one or more therapeutic agents administered to the subject were gemcitabine and fluorouracil. In some embodiments, the one or more therapeutic agents administered to the subject were paclitaxel and cisplatin. In some embodiments, the one or more therapeutic agents administered to the subject were paclitaxel and carboplatin. In some embodiments, the one or more therapeutic agents administered to the subject were paclitaxel and topotecan. In some embodiments, the one or more therapeutic agents administered to the subject was bevacizumab. In some embodiments, the one or more therapeutic agents administered to the subject was selected from the group consisting of a chemotherapeutic agent, pemetrexed, nab-paclitaxel, vinorelbine, bevacizumab, cisplatin, carboplatin, paclitaxel, topotecan, a combination of bevacizumab and paclitaxel, a combination of bevacizumab and cisplatin, a combination of bevacizumab and carboplatin, a combination of paclitaxel and topotecan, a combination of bevacizumab and topotecan, a combination of bevacizumab, cisplatin and paclitaxel, a combination of bevacizumab, carboplatin and paclitaxel, and a combination of bevacizumab, paclitaxel and topotecan. In some embodiments, the one or more therapeutic agents administered to the subject was a chemotherapeutic agent. In some embodiments, the one or more therapeutic agents administered to the subject was cisplatin, In some embodiments, the one or more therapeutic agents administered to the subject was carboplatin. In some embodiments, the one or more therapeutic agents administered to the subject was paclitaxel. In some embodiments, the one or more therapeutic agents administered to the subject was topotecan. In some embodiments, the one or more therapeutic agents administered to the subject was a combination of bevacizumab and paclitaxel. In some embodiments, the one or more therapeutic agents administered to the subject was a combination of bevacizumab and cisplatin. In some embodiments, the one or more therapeutic agents administered to the subject was a combination of bevacizumab and carboplatin. In some embodiments, the one or more therapeutic agents administered to the subject was a combination of paclitaxel and topotecan. In some embodiments, the one or more therapeutic agents administered to the subject was a combination of bevacizumab and topotecan. In some embodiments, the one or more therapeutic agents administered to the subject was a combination of bevacizumab, cisplatin and paclitaxel. In some embodiments, the one or more therapeutic agents administered to the subject was a combination of bevacizumab, carboplatin and paclitaxel. In some embodiments, the one or more therapeutic agents administered to the subject was a combination of bevacizumab, paclitaxel and topotecan. In some embodiments, the subject received treatment for the cervical cancer with irradiation and did not respond to the irradiation. In some embodiments, the subject did not respond to treatment with no more than two prior systemic treatment regiments. In some embodiments, the subject did not respond to treatment with one or two prior systemic treatment regimens. In some embodiments, the subject did not respond to treatment with one prior systemic treatment regimen. In some embodiments, the subject did not respond to treatment with two prior systemic treatment regimens.

In some embodiments of the methods or uses provided herein, the subject has been previously treated for the cervical cancer with one or more therapeutic agents. In some embodiments, the subject relapsed after the treatment. In some embodiments, the one or more therapeutic agents administered to the subject was not an anti-TF antibody-drug conjugate as described herein. In some embodiments, the one or more therapeutic agents administered to the subject was paclitaxel, cisplatin, carboplatin, topotecan, gemcitabine, fluorouracil, ixabepilone, imatinib mesylate, docetaxel, gefitinib, paclitaxel, pemetrexed, vinorelbine, doxil, cetuximab, pembrolizumab, nivolumab, bevacizumab, or any combination thereof. In some embodiments, the one or more therapeutic agents administered to the subject was a platinum-based therapeutic agent. In some embodiments, the one or more therapeutic agents administered to the subject were gemcitabine and fluorouracil. In some embodiments, the one or more therapeutic agents administered to the subject were paclitaxel and cisplatin. In some embodiments, the one or more therapeutic agents administered to the subject were paclitaxel and carboplatin. In some embodiments, the one or more therapeutic agents administered to the subject were paclitaxel and topotecan. In some embodiments, the one or more therapeutic agents administered to the subject was bevacizumab. In some embodiments, the one or more therapeutic agents administered to the subject was selected from the group consisting of a chemotherapeutic agent, pemetrexed, nab-paclitaxel, vinorelbine, bevacizumab, cisplatin, carboplatin, paclitaxel, topotecan, a combination of bevacizumab and paclitaxel, a combination of bevacizumab and cisplatin, a combination of bevacizumab and carboplatin, a combination of paclitaxel and topotecan, a combination of bevacizumab and topotecan, a combination of bevacizumab, cisplatin and paclitaxel, a combination of bevacizumab, carboplatin and paclitaxel, and a combination of bevacizumab, paclitaxel and topotecan. In some embodiments, the one or more therapeutic agents administered to the subject was a chemotherapeutic agent. In some embodiments, the one or more therapeutic agents administered to the subject was cisplatin, In some embodiments, the one or more therapeutic agents administered to the subject was carboplatin. In some embodiments, the one or more therapeutic agents administered to the subject was paclitaxel. In some embodiments, the one or more therapeutic agents administered to the subject was topotecan. In some embodiments, the one or more therapeutic agents administered to the subject was a combination of bevacizumab and paclitaxel. In some embodiments, the one or more therapeutic agents administered to the subject was a combination of bevacizumab and cisplatin. In some embodiments, the one or more therapeutic agents administered to the subject was a combination of bevacizumab and carboplatin. In some embodiments, the one or more therapeutic agents administered to the subject was a combination of paclitaxel and topotecan. In some embodiments, the one or more therapeutic agents administered to the subject was a combination of bevacizumab and topotecan. In some embodiments, the one or more therapeutic agents administered to the subject was a combination of bevacizumab, cisplatin and paclitaxel. In some embodiments, the one or more therapeutic agents administered to the subject was a combination of bevacizumab, carboplatin and paclitaxel. In some embodiments, the one or more therapeutic agents administered to the subject was a combination of bevacizumab, paclitaxel and topotecan. In some embodiments, the subject received treatment for the cervical cancer with irradiation and relapsed after treatment with irradiation. In some embodiments, the subject relapsed after treatment with no more than two prior systemic treatment regiments. In some embodiments, the subject relapsed after treatment with one or two prior systemic treatment regimens. In some embodiments, the subject relapsed after treatment with one prior systemic treatment regimen. In some embodiments, the subject relapsed after treatment with two prior systemic treatment regimens.

In some embodiments of the methods or uses provided herein, the subject has been previously treated for the cervical cancer with one or more therapeutic agents. In some embodiments, the subject experienced disease progression after the treatment. In some embodiments, the one or more therapeutic agents administered to the subject was not an anti-TF antibody-drug conjugate as described herein. In some embodiments, the one or more therapeutic agents administered to the subject was paclitaxel, cisplatin, carboplatin, topotecan, gemcitabine, fluorouracil, ixabepilone, imatinib mesylate, docetaxel, gefitinib, paclitaxel, pemetrexed, vinorelbine, doxil, cetuximab, pembrolizumab, nivolumab, bevacizumab, or any combination thereof. In some embodiments, the one or more therapeutic agents administered to the subject was a platinum-based therapeutic agent. In some embodiments, the one or more therapeutic agents administered to the subject were gemcitabine and fluorouracil. In some embodiments, the one or more therapeutic agents administered to the subject were paclitaxel and cisplatin. In some embodiments, the one or more therapeutic agents administered to the subject were paclitaxel and carboplatin. In some embodiments, the one or more therapeutic agents administered to the subject were paclitaxel and topotecan. In some embodiments, the one or more therapeutic agents administered to the subject was bevacizumab. In some embodiments, the one or more therapeutic agents administered to the subject was selected from the group consisting of a chemotherapeutic agent, pemetrexed, nab-paclitaxel, vinorelbine, bevacizumab, cisplatin, carboplatin, paclitaxel, topotecan, a combination of bevacizumab and paclitaxel, a combination of bevacizumab and cisplatin, a combination of bevacizumab and carboplatin, a combination of paclitaxel and topotecan, a combination of bevacizumab and topotecan, a combination of bevacizumab, cisplatin and paclitaxel, a combination of bevacizumab, carboplatin and paclitaxel, and a combination of bevacizumab, paclitaxel and topotecan. In some embodiments, the one or more therapeutic agents administered to the subject was a chemotherapeutic agent. In some embodiments, the one or more therapeutic agents administered to the subject was cisplatin, In some embodiments, the one or more therapeutic agents administered to the subject was carboplatin. In some embodiments, the one or more therapeutic agents administered to the subject was paclitaxel. In some embodiments, the one or more therapeutic agents administered to the subject was topotecan. In some embodiments, the one or more therapeutic agents administered to the subject was a combination of bevacizumab and paclitaxel. In some embodiments, the one or more therapeutic agents administered to the subject was a combination of bevacizumab and cisplatin. In some embodiments, the one or more therapeutic agents administered to the subject was a combination of bevacizumab and carboplatin. In some embodiments, the one or more therapeutic agents administered to the subject was a combination of paclitaxel and topotecan. In some embodiments, the one or more therapeutic agents administered to the subject was a combination of bevacizumab and topotecan. In some embodiments, the one or more therapeutic agents administered to the subject was a combination of bevacizumab, cisplatin and paclitaxel. In some embodiments, the one or more therapeutic agents administered to the subject was a combination of bevacizumab, carboplatin and paclitaxel. In some embodiments, the one or more therapeutic agents administered to the subject was a combination of bevacizumab, paclitaxel and topotecan. In some embodiments, the subject previously received treatment for the cervical cancer with irradiation and experienced disease progression after treatment with irradiation. In some embodiments, the subject experienced disease progression after treatment with no more than two prior systemic treatment regiments. In some embodiments, the subject experienced disease progression after treatment with one or two prior systemic treatment regimens. In some embodiments, the subject experienced disease progression after treatment with one prior systemic treatment regimen. In some embodiments, the subject experienced disease progression after treatment with two prior systemic treatment regimens.

B. Routes of Administration

An antibody-drug conjugate or antigen-binding fragment thereof described herein can be administered by any suitable route and mode. Suitable routes of administering antibody-drug conjugate of the present invention are well known in the art and may be selected by those of ordinary skill in the art. In one embodiment, the antibody-drug conjugate is administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of an antibody-drug conjugate or antigen-binding fragment described herein is intravenous injection or infusion. In some embodiments, the route of administration of an antibody-drug conjugate or antigen-binding fragment described herein is intravenous infusion.

C. Dosage and Frequency of Administration

In one aspect, the present invention provides for methods of treating a subject with cervical cancer as described herein with a particular dose of an antibody-drug conjugate or antigen-binding fragment thereof as described herein, wherein the subject is administered the antibody-drug conjugate or antigen-binding fragment thereof as described herein with a particular frequency.

In one embodiment of the methods or uses provided herein, an antibody-drug conjugate or antigen-binding fragment thereof as described herein is administered to the subject at a dose ranging from about 1.5 mg/kg to about 2.1 mg/kg of the subject's body weight. In certain embodiments, the dose is about 1.5 mg/kg, about 1.6 mg/kg, about 1.7 mg/kg, about 1.8 mg/kg, about 1.9 mg/kg, about 2.0 mg/kg or about 2.1 mg/kg. In one embodiment, the dose is about 2.0 mg/kg. In one embodiment, the dose is 2.0 mg/kg. In some embodiments, the dose is 2.0 mg/kg and the antibody-drug conjugate is tisotumab vedotin.

In one embodiment of the methods or uses or product for uses provided herein, an anti-TF antibody-drug conjugate or antigen-binding fragment thereof as described herein is administered to the subject at a dose ranging from about 0.65 mg/kg to about 2.1 mg/kg of the subject's body weight. In certain embodiments, the dose is about 0.65 mg/kg, about 0.7 mg/kg, about 0.75 mg/kg, about 0.8 mg/kg, about 0.85 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg or about 2.1 mg/kg. In one embodiment, the dose is about 0.65 mg/kg. In one embodiment, the dose is about 0.9 mg/kg. In one embodiment, the dose is about 1.3 mg/kg. In one embodiment, the dose is about 2.0 mg/kg. In certain embodiments, the dose is 0.65 mg/kg, 0.7 mg/kg, 0.75 mg/kg, 0.8 mg/kg, 0.85 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg or 2.1 mg/kg. In one embodiment, the dose is 0.65 mg/kg. In one embodiment, the dose is 0.9 mg/kg. In one embodiment, the dose is 1.3 mg/kg. In one embodiment, the dose is 2.0 mg/kg. In some embodiments, the dose is 0.65 mg/kg and the anti-TF antibody-drug conjugate is tisotumab vedotin. In some embodiments, the dose is 0.9 mg/kg and the anti-TF antibody-drug conjugate is tisotumab vedotin. In some embodiments, the dose is 1.3 mg/kg and the anti-TF antibody-drug conjugate is tisotumab vedotin. In some embodiments, the dose is 2.0 mg/kg and the anti-TF antibody-drug conjugate is tisotumab vedotin. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 65 mg, 90 mg, 130 mg, or 200 mg.

In one embodiment of the methods or uses provided herein, an antibody-drug conjugate or antigen-binding fragment thereof as described herein is administered to the subject once about every 1 to 4 weeks. In certain embodiments, the an antibody-drug conjugate or antigen-binding fragment thereof as described herein is administered once about every 1 week, once about every 2 weeks, once about every 3 weeks or once about every 4 weeks. In one embodiment, an antibody-drug conjugate or antigen-binding fragment thereof as described herein is administered once about every 3 weeks. In one embodiment, an antibody-drug conjugate or antigen-binding fragment thereof as described herein is administered once every 3 weeks. In some embodiments, the dose is about 0.65 mg/kg and is administered once about every 1 week. In some embodiments, the dose is about 0.65 mg/kg and is administered once about every 2 weeks. In some embodiments, the dose is about 0.65 mg/kg and is administered once about every 3 weeks. In some embodiments, the dose is about 0.65 mg/kg and is administered once about every 4 weeks. In some embodiments, the dose is about 0.7 mg/kg and is administered once about every 1 week. In some embodiments, the dose is about 0.7 mg/kg and is administered once about every 2 weeks. In some embodiments, the dose is about 0.7 mg/kg and is administered once about every 3 weeks. In some embodiments, the dose is about 0.7 mg/kg and is administered once about every 4 weeks. In some embodiments, the dose is about 0.75 mg/kg and is administered once about every 1 week. In some embodiments, the dose is about 0.75 mg/kg and is administered once about every 2 weeks. In some embodiments, the dose is about 0.75 mg/kg and is administered once about every 3 weeks. In some embodiments, the dose is about 0.75 mg/kg and is administered once about every 4 weeks. In some embodiments, the dose is about 0.8 mg/kg and is administered once about every 1 week. In some embodiments, the dose is about 0.8 mg/kg and is administered once about every 2 weeks. In some embodiments, the dose is about 0.8 mg/kg and is administered once about every 3 weeks. In some embodiments, the dose is about 0.8 mg/kg and is administered once about every 4 weeks. In some embodiments, the dose is about 0.85 mg/kg and is administered once about every 1 week. In some embodiments, the dose is about 0.85 mg/kg and is administered once about every 2 weeks. In some embodiments, the dose is about 0.85 mg/kg and is administered once about every 3 weeks. In some embodiments, the dose is about 0.85 mg/kg and is administered once about every 4 weeks. In some embodiments, the dose is about 0.9 mg/kg and is administered once about every 1 week. In some embodiments, the dose is about 0.9 mg/kg and is administered once about every 2 weeks. In some embodiments, the dose is about 0.9 mg/kg and is administered once about every 3 weeks. In some embodiments, the dose is about 0.9 mg/kg and is administered once about every 4 weeks. In some embodiments, the dose is about 1.0 mg/kg and is administered once about every 1 week. In some embodiments, the dose is about 1.0 mg/kg and is administered once about every 2 weeks. In some embodiments, the dose is about 1.0 mg/kg and is administered once about every 3 weeks. In some embodiments, the dose is about 1.0 mg/kg and is administered once about every 4 weeks. In some embodiments, the dose is about 1.1 mg/kg and is administered once about every 1 week. In some embodiments, the dose is about 1.1 mg/kg and is administered once about every 2 weeks. In some embodiments, the dose is about 1.1 mg/kg and is administered once about every 3 weeks. In some embodiments, the dose is about 1.1 mg/kg and is administered once about every 4 weeks. In some embodiments, the dose is about 1.2 mg/kg and is administered once about every 1 week. In some embodiments, the dose is about 1.2 mg/kg and is administered once about every 2 weeks. In some embodiments, the dose is about 1.2 mg/kg and is administered once about every 3 weeks. In some embodiments, the dose is about 1.2 mg/kg and is administered once about every 4 weeks. In some embodiments, the dose is about 1.3 mg/kg and is administered once about every 1 week. In some embodiments, the dose is about 1.3 mg/kg and is administered once about every 2 weeks. In some embodiments, the dose is about 1.3 mg/kg and is administered once about every 3 weeks. In some embodiments, the dose is about 1.3 mg/kg and is administered once about every 4 weeks. In some embodiments, the dose is about 1.4 mg/kg and is administered once about every 1 week. In some embodiments, the dose is about 1.4 mg/kg and is administered once about every 2 weeks. In some embodiments, the dose is about 1.4 mg/kg and is administered once about every 3 weeks. In some embodiments, the dose is about 1.4 mg/kg and is administered once about every 4 weeks. In some embodiments, the dose is about 1.5 mg/kg and is administered once about every 1 week. In some embodiments, the dose is about 1.5 mg/kg and is administered once about every 2 weeks. In some embodiments, the dose is about 1.5 mg/kg and is administered once about every 3 weeks. In some embodiments, the dose is about 1.5 mg/kg and is administered once about every 4 weeks. In some embodiments, the dose is about 1.6 mg/kg and is administered once about every 1 week. In some embodiments, the dose is about 1.6 mg/kg and is administered once about every 2 weeks. In some embodiments, the dose is about 1.6 mg/kg and is administered once about every 3 weeks. In some embodiments, the dose is about 1.6 mg/kg and is administered once about every 4 weeks. In some embodiments, the dose is about 1.7 mg/kg and is administered once about every 1 week. In some embodiments, the dose is about 1.7 mg/kg and is administered once about every 2 weeks. In some embodiments, the dose is about 1.7 mg/kg and is administered once about every 3 weeks. In some embodiments, the dose is about 1.7 mg/kg and is administered once about every 4 weeks. In some embodiments, the dose is about 1.8 mg/kg and is administered once about every 1 week. In some embodiments, the dose is about 1.8 mg/kg and is administered once about every 2 weeks. In some embodiments, the dose is about 1.8 mg/kg and is administered once about every 3 weeks. In some embodiments, the dose is about 1.8 mg/kg and is administered once about every 4 weeks. In some embodiments, the dose is about 1.9 mg/kg and is administered once about every 1 week. In some embodiments, the dose is about 1.9 mg/kg and is administered once about every 2 weeks. In some embodiments, the dose is about 1.9 mg/kg and is administered once about every 3 weeks. In some embodiments, the dose is about 1.9 mg/kg and is administered once about every 4 weeks. In some embodiments, the dose is about 2.0 mg/kg and is administered once about every 1 week. In some embodiments, the dose is about 2.0 mg/kg and is administered once about every 2 weeks. In some embodiments, the dose is about 2.0 mg/kg and is administered once about every 3 weeks. In some embodiments, the dose is about 2.0 mg/kg and is administered once about every 4 weeks. In some embodiments, the dose is about 2.1 mg/kg and is administered once about every 1 week. In some embodiments, the dose is about 2.1 mg/kg and is administered once about every 2 weeks. In some embodiments, the dose is about 2.1 mg/kg and is administered once about every 3 weeks. In some embodiments, the dose is about 2.1 mg/kg and is administered once about every 4 weeks. In some embodiments, the dose is 0.65 mg/kg and is administered once about every 1 week. In some embodiments, the dose is 0.65 mg/kg and is administered once about every 2 weeks. In some embodiments, the dose is 0.65 mg/kg and is administered once about every 3 weeks. In some embodiments, the dose is 0.65 mg/kg and is administered once about every 4 weeks. In some embodiments, the dose is 0.7 mg/kg and is administered once about every 1 week. In some embodiments, the dose is 0.7 mg/kg and is administered once about every 2 weeks. In some embodiments, the dose is 0.7 mg/kg and is administered once about every 3 weeks. In some embodiments, the dose is 0.7 mg/kg and is administered once about every 4 weeks. In some embodiments, the dose is 0.75 mg/kg and is administered once about every 1 week. In some embodiments, the dose is 0.75 mg/kg and is administered once about every 2 weeks. In some embodiments, the dose is 0.75 mg/kg and is administered once about every 3 weeks. In some embodiments, the dose is 0.75 mg/kg and is administered once about every 4 weeks. In some embodiments, the dose is 0.8 mg/kg and is administered once about every 1 week. In some embodiments, the dose is 0.8 mg/kg and is administered once about every 2 weeks. In some embodiments, the dose is 0.8 mg/kg and is administered once about every 3 weeks. In some embodiments, the dose is 0.8 mg/kg and is administered once about every 4 weeks. In some embodiments, the dose is 0.85 mg/kg and is administered once about every 1 week. In some embodiments, the dose is 0.85 mg/kg and is administered once about every 2 weeks. In some embodiments, the dose is 0.85 mg/kg and is administered once about every 3 weeks. In some embodiments, the dose is 0.85 mg/kg and is administered once about every 4 weeks. In some embodiments, the dose is 0.9 mg/kg and is administered once about every 1 week. In some embodiments, the dose is 0.9 mg/kg and is administered once about every 2 weeks. In some embodiments, the dose is 0.9 mg/kg and is administered once about every 3 weeks. In some embodiments, the dose is 0.9 mg/kg and is administered once about every 4 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered once about every 3 weeks. In some embodiments, the dose is 1.0 mg/kg and is administered once about every 4 weeks. In some embodiments, the dose is 1.1 mg/kg and is administered once about every 1 week. In some embodiments, the dose is 1.1 mg/kg and is administered once about every 2 weeks. In some embodiments, the dose is 1.1 mg/kg and is administered once about every 3 weeks. In some embodiments, the dose is 1.1 mg/kg and is administered once about every 4 weeks. In some embodiments, the dose is 1.2 mg/kg and is administered once about every 1 week. In some embodiments, the dose is 1.2 mg/kg and is administered once about every 2 weeks. In some embodiments, the dose is 1.2 mg/kg and is administered once about every 3 weeks. In some embodiments, the dose is 1.2 mg/kg and is administered once about every 4 weeks. In some embodiments, the dose is 1.3 mg/kg and is administered once about every 1 week. In some embodiments, the dose is 1.3 mg/kg and is administered once about every 2 weeks. In some embodiments, the dose is 1.3 mg/kg and is administered once about every 3 weeks. In some embodiments, the dose is 1.3 mg/kg and is administered once about every 4 weeks. In some embodiments, the dose is 1.4 mg/kg and is administered once about every 1 week. In some embodiments, the dose is 1.4 mg/kg and is administered once about every 2 weeks. In some embodiments, the dose is 1.4 mg/kg and is administered once about every 3 weeks. In some embodiments, the dose is 1.4 mg/kg and is administered once about every 4 weeks. In some embodiments, the dose is 1.5 mg/kg and is administered once about every 1 week. In some embodiments, the dose is 1.5 mg/kg and is administered once about every 2 weeks. In some embodiments, the dose is 1.5 mg/kg and is administered once about every 3 weeks. In some embodiments, the dose is 1.5 mg/kg and is administered once about every 4 weeks. In some embodiments, the dose is 1.6 mg/kg and is administered once about every 1 week. In some embodiments, the dose is 1.6 mg/kg and is administered once about every 2 weeks. In some embodiments, the dose is 1.6 mg/kg and is administered once about every 3 weeks. In some embodiments, the dose is 1.6 mg/kg and is administered once about every 4 weeks. In some embodiments, the dose is 1.7 mg/kg and is administered once about every 1 week. In some embodiments, the dose is 1.7 mg/kg and is administered once about every 2 weeks. In some embodiments, the dose is 1.7 mg/kg and is administered once about every 3 weeks. In some embodiments, the dose is 1.7 mg/kg and is administered once about every 4 weeks. In some embodiments, the dose is 1.8 mg/kg and is administered once about every 1 week. In some embodiments, the dose is 1.8 mg/kg and is administered once about every 2 weeks. In some embodiments, the dose is 1.8 mg/kg and is administered once about every 3 weeks. In some embodiments, the dose is 1.8 mg/kg and is administered once about every 4 weeks. In some embodiments, the dose is 1.9 mg/kg and is administered once about every 1 week. In some embodiments, the dose is 1.9 mg/kg and is administered once about every 2 weeks. In some embodiments, the dose is 1.9 mg/kg and is administered once about every 3 weeks. In some embodiments, the dose is 1.9 mg/kg and is administered once about every 4 weeks. In some embodiments, the dose is 2.0 mg/kg and is administered once about every 1 week. In some embodiments, the dose is 2.0 mg/kg and is administered once about every 2 weeks. In some embodiments, the dose is 2.0 mg/kg and is administered once about every 3 weeks. In some embodiments, the dose is 2.0 mg/kg and is administered once about every 4 weeks. In some embodiments, the dose is 2.1 mg/kg and is administered once about every 1 week. In some embodiments, the dose is 2.1 mg/kg and is administered once about every 2 weeks. In some embodiments, the dose is 2.1 mg/kg and is administered once about every 3 weeks. In some embodiments, the dose is 2.1 mg/kg and is administered once about every 4 weeks. In some embodiments, the dose is 2.0 mg/kg and is administered once about every 3 weeks (e.g., ±3 days). In some embodiments, the dose is 2.0 mg/kg and is administered once every 3 weeks. In some embodiments, the dose is 2.0 mg/kg and is administered once every 3 weeks and the antibody-drug conjugate is tisotumab vedotin. In some embodiments, the dose is 2.0 mg/kg and is administered once every 3 weeks and the antibody-drug conjugate is tisotumab vedotin and the dose is decreased to 1.3 mg/kg if one or more adverse events occur. In some embodiments, the dose is 1.3 mg/kg and is administered once every 3 weeks. In some embodiments, the dose is 1.3 mg/kg and is administered once every 3 weeks and the antibody-drug conjugate is tisotumab vedotin. In some embodiments, the dose is 1.3 mg/kg and is administered once every 3 weeks and the antibody-drug conjugate is tisotumab vedotin and the dose is decreased to 0.9 mg/kg if one or more adverse events occur. In some embodiments, the dose is about 0.9 mg/kg and is administered once about every week and the antibody-drug conjugate is tisotumab vedotin. In some embodiments, the dose is 0.9 mg/kg and is administered once every week and the antibody-drug conjugate is tisotumab vedotin. In some embodiments, the dose is about 0.65 mg/kg and is administered once about every week and the antibody-drug conjugate is tisotumab vedotin. In some embodiments, the dose is 0.65 mg/kg and is administered once every week and the antibody-drug conjugate is tisotumab vedotin. In some embodiments, for a subject weighing more than 100 kg, the dose of the anti-TF antibody-drug conjugate administered is the amount that would be administered if the subject weighed 100 kg. In some embodiments, for a subject weighing more than 100 kg, the dose of the anti-TF antibody-drug conjugate administered is 65 mg, 90 mg, 130 mg, or 200 mg.

In one embodiment of the methods or uses provided herein, an antibody-drug conjugate or antigen-binding fragment thereof as described herein is administered to the subject at a fixed dose of between 50 mg and 200 mg such as at a dose of 50 mg or a dose of 60 mg or a dose of 70 mg or a dose of 80 mg or a dose of 90 mg or a dose of 100 mg or a dose of 110 mg or a dose of 120 mg or a dose of 130 mg or a dose of 140 mg or a dose of 150 mg or a dose of 160 mg or a dose of 170 mg or a dose of 180 mg or a dose of 190 mg or a dose of 200 mg. In some embodiments, the fixed dose is administered to the subject once about every 1 to 4 weeks. In certain embodiments, the fixed dose is administered to the subject once about every 1 week, once about every 2 weeks, once about every 3 weeks or once about every 4 weeks. In some embodiments, the fixed dose is administered to the subject once about every 3 weeks (e.g., ±3 days). In some embodiments, the fixed dose is administered to the subject once every 3 weeks. In some embodiments, the fixed dose is administered to the subject once every 3 weeks and the antibody-drug conjugate is tisotumab vedotin.

In one embodiment of the methods or uses provided herein, an antibody-drug conjugate or antigen-binding fragment thereof as described herein is administered to the subject at a flat dose of between 50 mg and 200 mg such as at a dose of 50 mg or a dose of 60 mg or a dose of 70 mg or a dose of 80 mg or a dose of 90 mg or a dose of 100 mg or a dose of 110 mg or a dose of 120 mg or a dose of 130 mg or a dose of 140 mg or a dose of 150 mg or a dose of 160 mg or a dose of 170 mg or a dose of 180 mg or a dose of 190 mg or a dose of 200 mg. In some embodiments, the fixed dose is administered to the subject once about every 1 to 4 weeks. In certain embodiments, the fixed dose is administered to the subject once about every 1 week, once about every 2 weeks, once about every 3 weeks or once about every 4 weeks. In some embodiments, the fixed dose is administered to the subject once about every 3 weeks (e.g., ±3 days). In some embodiments, the fixed dose is administered to the subject once every 3 weeks. In some embodiments, the fixed dose is administered to the subject once every 3 weeks and the antibody-drug conjugate is tisotumab vedotin.

In some embodiments, a method of treatment or use described herein further comprises the administration of one or more additional therapeutic agents. In some embodiments, the one or more additional therapeutic agents are administered simultaneously with an antibody-drug conjugate or antigen-binding fragment thereof as described herein, such as tisotumab vedotin. In some embodiments, the one or more additional therapeutic agents and an antibody-drug conjugate or antigen-binding fragment thereof as described herein are administered sequentially.

D. Treatment Outcome

In one aspect, a method of treating cervical cancer with an antibody-drug conjugates or antigen-binding fragments thereof described herein results in an improvement in one or more therapeutic effects in the subject after administration of the antibody-drug conjugate relative to a baseline. In some embodiments, the one or more therapeutic effects is the size of the tumor derived from the cervical cancer, the objective response rate, the duration of response, the time to response, progression free survival, overall survival, or any combination thereof. In one embodiment, the one or more therapeutic effects is the size of the tumor derived from the cervical cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses provided herein, response to treatment with an antibody-drug conjugate or antigen-binding fragment thereof described herein may include the following criteria (RECIST Criteria 1.1):

| | Category | Criteria |
|---|---|---|
| Based on | Complete | Disappearance of all target lesions. Any |
| target lesions | Response (CR) | pathological lymph nodes must have reduction |
| | | in short axis to <10 mm. |
| | Partial Response | ≥30% decrease in the sum of the longest diameter |
| | (PR) | (LD) of target lesions, taking as reference the |
| | | baseline sum of LDs. |
| | Stable Disease | Neither sufficient shrinkage to qualify for PR nor |
| | (SD) | sufficient increase to qualify for PD, taking as |
| | | reference the smallest sum of LDs while in trial. |
| | Progressive | ≥20% (and ≥5 mm) increase in the sum of the LDs |
| | Disease (PD) | of target lesions, taking as reference the smallest |
| | | sum of the target LDs recorded while in trial or |
| | | the appearance of one or more new lesions. |
| Based on non- | CR | Disappearance of all non-target lesions and |
| target lesions | | normalization of tumor marker level. All |
| | | lymph nodes must be non-pathological in size |
| | | (<10 mm short axis). |
| | SD | Persistence of one or more non-target lesion(s) |
| | | or/and maintenance of tumor marker level above |
| | | the normal limits. |
| | PD | Appearance of one or more new lesions and/or |
| | | unequivocal progression of existing non-target |
| | | lesions. |

In one embodiment of the methods or uses provided herein, the effectiveness of treatment with an antibody-drug conjugate or antigen-binding fragment thereof described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is 100%.

In one embodiment of the methods or uses provided herein, response to treatment with an antibody-drug conjugate or antigen-binding fragment thereof described herein is assessed by measuring the size of a tumor derived from the cervical cancer. In one embodiment, the size of a tumor derived from the cervical cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cervical cancer before administration of the antibody-drug conjugate. In one embodiment, the size of a tumor derived from the cervical cancer is reduced by at least about 10%-80%. In one embodiment, the size of a tumor derived from the cervical cancer is reduced by at least about 20%-80%. In one embodiment, the size of a tumor derived from the cervical cancer is reduced by at least about 30%-80%. In one embodiment, the size of a tumor derived from the cervical cancer is reduced by at least about 40%-80%. In one embodiment, the size of a tumor derived from the cervical cancer is reduced by at least about 50%-80%. In one embodiment, the size of a tumor derived from the cervical cancer is reduced by at least about 60%-80%. In one embodiment, the size of a tumor derived from the cervical cancer is reduced by at least about 70%-80%. In one embodiment, the size of a tumor derived from the cervical cancer is reduced by at least about 80%. In one embodiment, the size of a tumor derived from the cervical cancer is reduced by at least about 85%. In one embodiment, the size of a tumor derived from the cervical cancer is reduced by at least about 90%. In one embodiment, the size of a tumor derived from the cervical cancer is reduced by at least about 95%. In one embodiment, the size of a tumor derived from the cervical cancer is reduced by at least about 98%. In one embodiment, the size of a tumor derived from the cervical cancer is reduced by at least about 99%. In one embodiment, the size of a tumor derived from the cervical cancer is reduced by 100%. In one embodiment, the size of a tumor derived from the cervical cancer is measured by magnetic resonance imaging (MRI). In one embodiment, the size of a tumor derived from the cervical cancer is measured by computed tomography (CT). In some embodiments, the size of a tumor derived from the cervical cancer is measured by pelvic examination. See Choi et al., 2008, J. Gynecol. Oncol. 19(3):205.

In one embodiment of the methods or uses provided described herein, response to treatment with an antibody-drug conjugate or antigen-binding fragment thereof described herein, such as e.g., tisotumab vedotin, promotes regression of a tumor derived from the cervical cancer. In one embodiment, a tumor derived from the cervical cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cervical cancer before administration of the antibody-drug conjugate. In one embodiment, a tumor derived from the cervical cancer regresses by at least about 10%-80%. In one embodiment, a tumor derived from the cervical cancer regresses by at least about 20%-80%. In one embodiment, a tumor derived from the cervical cancer regresses by at least about 30%-80%. In one embodiment, a tumor derived from the cervical cancer regresses by at least about 40%-80%. In one embodiment, a tumor derived from the cervical cancer regresses by at least about 50%-80%. In one embodiment, a tumor derived from the cervical cancer regresses by at least about 60%-80%. In one embodiment, a tumor derived from the cervical cancer regresses by at least about 70%-80%. In one embodiment, a tumor derived from the cervical cancer regresses by at least about 80%. In one embodiment, a tumor derived from the cervical cancer regresses by at least about 85%. In one embodiment, a tumor derived from the cervical cancer regresses by at least about 90%. In one embodiment, a tumor derived from the cervical cancer regresses by at least about 95%. In one embodiment, a tumor derived from the cervical cancer regresses by at least about 98%. In one embodiment, a tumor derived from the cervical cancer regresses by at least about 99%. In one embodiment, a tumor derived from the cervical cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In some embodiments, regression of a tumor is determined by measuring the size of the tumor by pelvic examination. See Choi et al., 2008, J. Gynecol. Oncol. 19(3):205.

In some embodiments of the methods or uses provided herein, response to treatment with an antibody-drug conjugate or antigen-binding fragment thereof described herein promotes regression of the number of tumors derived from the cervical cancer. In some embodiments, regression of the number of tumors is determined by detecting the number of tumors in the subject by Mill, CT scan, or pelvic examination. See Choi et al., 2008, J. Gynecol. Oncol. 19(3):205.

In one embodiment of the methods or uses described herein, response to treatment with an antibody-drug conjugate or antigen-binding fragment thereof described herein is assessed by measuring the time of progression free survival after administration of the antibody-drug conjugate. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the subject exhibits progression-free survival of at least about 6 months after administration of the antibody-drug conjugate. In some embodiments, the subject exhibits progression-free survival of at least about one year after administration of the antibody-drug conjugate. In some embodiments, the subject exhibits progression-free survival of at least about two years after administration of the antibody-drug conjugate. In some embodiments, the subject exhibits progression-free survival of at least about three years after administration of the antibody-drug conjugate. In some embodiments, the subject exhibits progression-free survival of at least about four years after administration of the antibody-drug conjugate. In some embodiments, the subject exhibits progression-free survival of at least about five years after administration of the antibody-drug conjugate.

In one embodiment of the methods or uses described herein, response to treatment with an antibody-drug conjugate or antigen-binding fragment thereof described herein is assessed by measuring the time of overall survival after administration of the antibody-drug conjugate. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the subject exhibits overall survival of at least about 6 months after administration of the antibody-drug conjugate. In some embodiments, the subject exhibits overall survival of at least about one year after administration of the antibody-drug conjugate. In some embodiments, the subject exhibits overall survival of at least about two years after administration of the antibody-drug conjugate. In some embodiments, the subject exhibits overall survival of at least about three years after administration of the antibody-drug conjugate. In some embodiments, the subject exhibits overall survival of at least about four years after administration of the antibody-drug conjugate. In some embodiments, the subject exhibits overall survival of at least about five years after administration of the antibody-drug conjugate.

In one embodiment of the methods or uses described herein, response to treatment with an antibody-drug conjugate or antigen-binding fragment thereof described herein is assessed by measuring the duration of response to the antibody-drug conjugate after administration of the antibody-drug conjugate. In some embodiments, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate. In some embodiments, the duration of response to the antibody-drug conjugate is at least about 6 months after administration of the antibody-drug conjugate. In some embodiments, the duration of response to the antibody-drug conjugate is at least about one year after administration of the antibody-drug conjugate. In some embodiments, the duration of response to the antibody-drug conjugate is at least about two years after administration of the antibody-drug conjugate. In some embodiments, the duration of response to the antibody-drug conjugate is at least about three years after administration of the antibody-drug conjugate. In some embodiments, the duration of response to the antibody-drug conjugate is at least about four years after administration of the antibody-drug conjugate. In some embodiments, the duration of response to the antibody-drug conjugate is at least about five years after administration of the antibody-drug conjugate.

E. Adverse Events

In one aspect, a method of treating cervical cancer with an antibody-drug conjugates or antigen-binding fragments thereof described herein results in the subject developing one or more adverse events. In some embodiments, the subject is administered an additional therapeutic agent to eliminate or reduce the severity of the adverse event. In some embodiments, the one or more adverse events the subject develops is anemia, abdominal pain, hypokalemia, hyponatremia, epistaxis, fatigue, nausea, alopecia, conjunctivitis, constipation, decreased appetite, diarrhea, vomiting, peripheral neuropathy, general physical health deterioration, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event. In some embodiments, the one or more adverse events is conjunctivitis and/or keratitis and the additional therapeutic agent is a preservative-free lubricating eye drop, an ocular vasoconstrictor, a steroid eye drop, or any combination thereof. In some embodiments, the one or more adverse events is conjunctivitis and keratitis and the additional therapeutic agent is a preservative-free lubricating eye drop, an ocular vasoconstrictor, a steroid eye drop, or any combination thereof. In some embodiments, the one or more adverse events is conjunctivitis and the additional therapeutic agent is a preservative-free lubricating eye drop, an ocular vasoconstrictor, a steroid eye drop, or any combination thereof. In some embodiments, the one or more adverse events is keratitis and the additional therapeutic agent is a preservative-free lubricating eye drop, an ocular vasoconstrictor, a steroid eye drop, or any combination thereof. In some of any of the embodiments herein, the subject is administered a treatment with or with the additional therapeutic agent to eliminate or reduce the severity of the adverse event (e.g., conjunctivitis and/or keratitis). In some embodiments, the treatment is eye cooling pads (e.g. THERA PEARL Eye Mask or similar), In some embodiments, the one or more adverse events is a recurrent infusion related reaction and the additional therapeutic agent is an antihistamine, acetaminophen and/or a corticosteroid. In some embodiments, the one or more adverse events is neutropenia and the additional therapeutic agent is growth factor support (G-CSF).

In one aspect, the subject treated with an antibody-drug conjugates or antigen-binding fragments thereof described herein is at risk of developing one or more adverse events. In some embodiments, the subject is administered an additional therapeutic agent to prevent the development of the adverse event or to reduce the severity of the adverse event. In some embodiments, the one or more adverse events the subject is at risk of developing is anemia, abdominal pain, hypokalemia, hyponatremia, epistaxis, fatigue, nausea, alopecia, conjunctivitis, constipation, decreased appetite, diarrhea, vomiting, peripheral neuropathy, general physical health deterioration, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event or. In some embodiments, the one or more adverse events is conjunctivitis and/or keratitis and the additional agent is a preservative-free lubricating eye drop, an ocular vasoconstrictor, a steroid eye drop, or any combination thereof. In some embodiments, the one or more adverse events is conjunctivitis and keratitis and the additional agent is a preservative-free lubricating eye drop, an ocular vasoconstrictor, a steroid eye drop, or any combination thereof. In some embodiments, the one or more adverse events is conjunctivitis and the additional agent is a preservative-free lubricating eye drop, an ocular vasoconstrictor, a steroid eye drop, or any combination thereof. In some embodiments, the one or more adverse events is keratitis and the additional agent is a preservative-free lubricating eye drop, an ocular vasoconstrictor, a steroid eye drop, or any combination thereof. In some of any of the embodiments herein, the subject is administered a treatment with or with the additional therapeutic agent to prevent the development of the adverse event or to reduce the severity of the adverse event (e.g., conjunctivitis and/or keratitis). In some embodiments, the treatment is eye cooling pads (e.g. THERA PEARL Eye Mask or similar), In some embodiments, the one or more adverse events is a recurrent infusion related reaction and the additional agent is an antihistamine, acetaminophen and/or a corticosteroid. In some embodiments, the one or more adverse events is neutropenia and the additional agent is growth factor support (G-CSF).

### IV. Compositions

In some aspects, also provided herein are compositions (e.g., pharmaceutical composition) comprising any of the anti-TF antibody-drug conjugates described herein.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.1% to about 25% by weight or between about 1 to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

Formulations comprising an anti-TF antibody-conjugate described herein for use in methods of treatment provided herein are described in WO2015/075201. In some embodiments, an anti-TF antibody-drug conjugate described herein is in a formulation comprising the anti-TF antibody drug conjugate, histidine, sucrose, and D-mannitol, wherein the formulation has a pH of about 6.0. In some embodiments, an anti-TF antibody-drug conjugate described herein is in a formulation comprising the anti-TF antibody drug conjugate at a concentration of about 10 mg/ml, histidine at a concentration of about 30 mM, sucrose at a concentration of about 88 mM, D-mannitol at a concentration of about 165 mM, wherein the formulation has a pH of about 6.0. In some embodiments, an anti-TF antibody-drug conjugate described herein is in a formulation comprising the anti-TF antibody drug conjugate at a concentration of 10 mg/ml, histidine at a concentration of 30 mM, sucrose at a concentration of 88 mM, D-mannitol at a concentration of 165 mM, wherein the formulation has a pH of 6.0. In some embodiments, the formulation comprises tisotumab vedotin at a concentration of 10 mg/ml, histidine at a concentration of 30 mM, sucrose at a concentration of 88 mM, D-mannitol at a concentration of 165 mM, wherein the formulation has a pH of 6.0.

In some embodiments provided herein, a formulation comprising the anti-TF antibody-conjugate described herein does not comprise a surfactant (i.e., is free of surfactant).

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The invention provides compositions comprising a population of anti-TF antibody-drug conjugates or antigen-binding fragments thereof as described herein for use in a method of treating cervical cancer as described herein. In some aspects, provided herein are compositions comprising a population of antibody-drug conjugates, wherein the antibody-drug conjugates comprise a linker attached to MMAE, wherein the antibody-drug conjugate has the following structure:

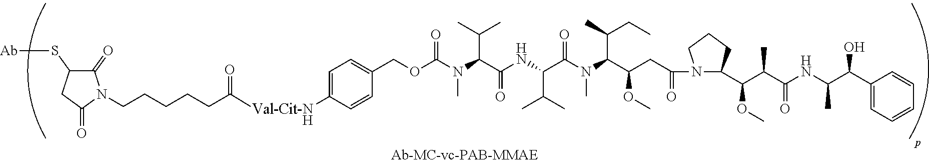

wherein p denotes a number from 1 to 8, S represents a sulphydryl residue of the anti-TF antibody or antigen-binding fragment thereof, and Ab designates the anti-TF antibody or antigen-binding fragment thereof as described herein, such as tisotumab. In some embodiments, p denotes a number from 3 to 5. In some embodiments, the average value of p in the composition is about 4. In some embodiments, the population is a mixed population of antibody-drug conjugates in which p varies from 1 to 8 for each antibody-drug conjugate. In some embodiments, the population is a homogenous population of antibody-drug conjugates with each antibody-drug conjugate having the same value for p.

In some embodiments, a composition comprising an antibody-drug conjugate as described herein is coadministered with one or additional therapeutic agents. In some embodiments the coadministration is simultaneous or sequential. In some embodiments, the antibody-drug conjugate as described herein is administered simultaneously with the one or more additional therapeutic agents. In some embodiments, simultaneous means that the antibody-drug conjugate and the one or more therapeutic agents are administered to the subject less than one hour apart, such as less than about 30 minutes apart, less than about 15 minutes apart, less than about 10 minutes apart or less than about 5 minutes apart. In some embodiments, the antibody-drug conjugate as described herein is administered sequentially with the one or more additional therapeutic agents. In some embodiments, sequential administration means that the antibody-drug conjugate and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, a composition comprising an antibody-drug conjugate as described herein is coadministered with one or more therapeutic agents to eliminate or reduce the severity of one or more adverse events. In some embodiments, a composition comprising an antibody-drug conjugate as described herein is coadministered with one or more therapeutic agents to prevent the development of the adverse event or to reduce the severity of the adverse event.

In some embodiments, a composition comprising an antibody-drug conjugate as described herein is coadministered with one or more therapeutic agents to eliminate or reduce the severity of one or more adverse events. In some embodiments the coadministration is simultaneous or sequential. In some embodiments, the antibody-drug conjugate as described herein is administered simultaneously with the one or more therapeutic agents to eliminate or reduce the severity of one or more adverse events. In some embodiments, simultaneous means that the antibody-drug conjugate and the one or more therapeutic agents to eliminate or reduce the severity of one or more adverse events are administered to the subject less than one hour apart, such as less than about 30 minutes apart, less than about 15 minutes apart, less than about 10 minutes apart or less than about 5 minutes apart. In some embodiments, the antibody-drug conjugate as described herein is administered sequentially with the one or more therapeutic agents to eliminate or reduce the severity of one or more adverse events. In some embodiments, sequential administration means that the antibody-drug conjugate and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the antibody-drug conjugate is administered prior to the one or more therapeutic agents to eliminate or reduce the severity of one or more adverse events. In some embodiments, the one or more therapeutic agents to eliminate or reduce the severity of one or more adverse events is administered prior to the antibody-drug conjugate.

### V. Articles of Manufacture and Kits

In another aspect, an article of manufacture or kit is provided which comprises an anti-TF antibody-drug conjugate described herein. The article of manufacture or kit may further comprise instructions for use of the antibody in the methods of the invention. Thus, in certain embodiments, the article of manufacture or kit comprises instructions for the use of an anti-TF antibody-drug conjugate in methods for treating cervical cancer in a subject comprising administering to the subject an effective amount of an anti-TF antibody-drug conjugate. In some embodiments, the cervical cancer is advanced cervical cancer, such as grade 3 cervical cancer or grade 4 cervical cancer. In some embodiments, the advanced cervical cancer is metastatic cancer. In some embodiments, the cervical cancer is metastatic cancer and recurrent cancer. In some embodiments the cervical cancer is recurrent cancer. In some embodiments, the subject has been previously treated with one or more therapeutic agents and did not respond to the treatment, relapsed after treatment, or experienced disease progression during treatment. In some embodiments herein of the previous treatment, the one or more therapeutic agents is not the antibody-drug conjugate. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating cervical cancer in a subject such as cervical cancer described herein (e.g., advanced cervical cancer such as grade 3 or grade 4 or metastatic cervical cancer). The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein the anti-TF antibody-drug conjugate is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein.

The article of manufacture or kit herein optionally further comprises a container comprising a second medicament, wherein the second medicament is for eliminating or reducing the severity of one or more adverse events, wherein the anti-TF antibody-drug conjugate is a first medicament, and which article or kit further comprises instructions on the label or package insert for treating the subject with the second medicament, in an effective amount. In some embodiments, the label or package insert indicates that the first and second medicaments are to be administered sequentially or simultaneously, as described herein, for example wherein the label or package insert indicates that the anti-TF antibody-drug conjugate is to be administered first, followed by administration of the second medicament.

In some embodiments, the anti-TF antibody-drug conjugate is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

### VI. Exemplary Embodiments

Among the embodiments provided herein are:

1. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 1.5 mg/kg to about 2.1 mg/kg.
2. The method of embodiment 1, wherein the dose is about 2.0 mg/kg.
3. The method of embodiment 1 or embodiment 2, wherein the antibody-drug conjugate is administered once about every 1 week, 2 weeks, 3 weeks or 4 weeks.
4. The method of any one of embodiments 1-3, wherein the antibody-drug conjugate is administered once about every 3 weeks.
5. The method of any one of embodiments 1-4, wherein the subject has been previously treated with one or more therapeutic agents and did not respond to the treatment, wherein the one or more therapeutic agents is not the antibody-drug conjugate.
6. The method of any one of embodiments 1-4, wherein the subject has been previously treated with one or more therapeutic agents and relapsed after the treatment, wherein the one or more therapeutic agents is not the antibody-drug conjugate.
7. The method of any one of embodiments 1-4, wherein the subject has been previously treated with one or more therapeutic agents and has experienced disease progression during the treatment, wherein the one or more therapeutic agents is not the antibody-drug conjugate.
8. The method of any one of embodiments 5-7, wherein the one or more therapeutic agents comprises a platinum-based therapeutic agent.
9. The method of any one of embodiments 5-7, wherein the one or more therapeutic agents is selected from the group consisting of: paclitaxel, cisplatin, carboplatin, topotecan, gemcitabine, fluorouracil, ixabepilone, imatinib mesylate, docetaxel, gefitinib, paclitaxel, pemetrexed, vinorelbine, doxil, cetuximab, pembrolizumab, nivolumab and bevacizumab.
10. The method of any one of embodiments 1-9, wherein the subject has experienced disease progression during or after treatment with:

a) paclitaxel and cisplatin,

b) paclitaxel and carboplatin, or

c) paclitaxel and topotecan.

11. The method of any one of embodiments 1-10, wherein the subject has received treatment with bevacizumab.
12. The method of any one of embodiments 1-10, wherein the subject is ineligible for treatment with bevacizumab.
13. The method of any one of embodiments 1-12, wherein the subject is not a candidate for curative therapy.
14. The method of embodiment 13, wherein the curative therapy comprises radiotherapy and/or exenterative surgery.
15. The method of any one of embodiments 1-14, wherein the subject did not respond to treatment with no more than two prior systemic treatment regimens.
16. The method of any one of embodiments 1-14, wherein the subject relapsed after treatment with no more than two prior systemic treatment regimens.
17. The method of any one of embodiments 1-16, wherein the cervical cancer is an adenocarcinoma, an adenosquamous carcinoma or a squamous cell carcinoma.
18. The method of any one of embodiments 1-17, wherein the cervical cancer is an advanced stage cervical cancer, such as a stage 3 or stage 4 cervical cancer, such as metastatic cervical cancer.
19. The method of any one of embodiments 1-18, wherein the cervical cancer is recurrent cervical cancer.
20. The method of any one of embodiments 1-19, wherein the monomethyl auristatin is monomethyl auristatin E (MMAE).
21. The method of any one of embodiments 1-20, wherein the anti-TF antibody or antigen-binding fragment thereof of the antibody-drug conjugate is a monoclonal antibody or a monoclonal antigen-binding fragment thereof.
22. The method of any one of embodiments 1-21, wherein the anti-TF antibody or antigen-binding fragment thereof of the antibody-drug conjugate comprises a heavy chain variable region and a light chain variable region, wherein the heavy chain variable region comprises:

(i) a CDR-H1 comprising the amino acid sequence of SEQ ID NO:1;

(ii) a CDR-H2 comprising the amino acid sequence of SEQ ID NO:2; and

(iii) a CDR-H3 comprising the amino acid sequence of SEQ ID NO:3; and

wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof of the antibody-drug conjugate are defined by the IMGT numbering scheme.

23. The method of any one of embodiments 1-22, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
24. The method of any one of embodiments 1-23, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
25. The method of any one of embodiments 1-24, wherein the anti-TF antibody of the antibody-drug conjugate is tisotumab.
26. The method of any one of embodiments 1-25, wherein the antibody-drug conjugate further comprises a linker between the anti-TF antibody or antigen-binding fragment thereof and the monomethyl auristatin.
27. The method of embodiment 26, wherein the linker is a cleavable peptide linker.
28. The method of embodiment 27, wherein the cleavable peptide linker has a formula: -MC-vc-PAB-, wherein:

### a) MC is:

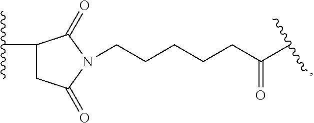 [SMILES: *C(=O)CCCCCN1C(=O)CC(*)C1=O]

b) vc is the dipeptide valine-citrulline, and

### c) PAB is:

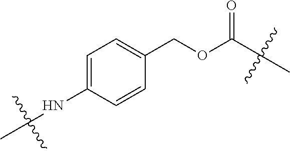 [SMILES: *Nc1ccc(COC(*)=O)cc1]

29. The method of any one of embodiments 26-28, wherein the linker is attached to sulphydryl residues of the anti-TF antibody obtained by partial reduction or full reduction of the anti-TF antibody or antigen-binding fragment thereof.
30. The method of embodiment 29, wherein the linker is attached to MMAE, wherein the antibody-drug conjugate has the following structure:

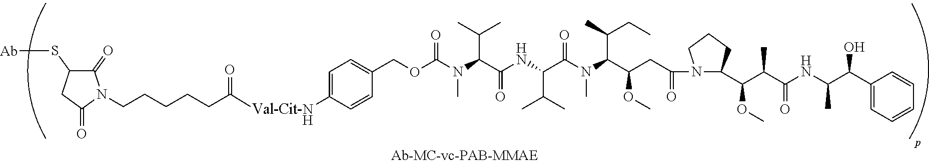

wherein p denotes a number from 1 to 8, S represents a sulphydryl residue of the anti-TF antibody, and Ab designates the anti-TF antibody or antigen-binding fragment thereof.
31. The method of embodiment 30, wherein the average value of p in a population of the antibody-drug conjugates is about 4.
32. The method of any one of embodiments 1-31, wherein the antibody-drug conjugate is tisotumab vedotin.
33. The method of any one of embodiments 1-32, wherein the route of administration for the antibody-drug conjugate is intravenous.
34. The method of any one of embodiments 1-33, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF.
35. The method of any one of embodiments 1-34, wherein one or more therapeutic effects in the subject is improved after administration of the antibody-drug conjugate relative to a baseline.
36. The method of embodiment 35, wherein the one or more therapeutic effects is selected from the group consisting of: size of a tumor derived from the cervical cancer, objective response rate, duration of response, time to response, progression free survival, and overall survival.
37. The method of any one of embodiments 1-36, wherein the size of a tumor derived from the cervical cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cervical cancer before administration of the antibody-drug conjugate.
38. The method of any one of embodiments 1-37, wherein the objective response rate is at least about 20%, at least about 25%, at least about 30%, at least about 35%, at least about 40%, at least about 45%, at least about 50%, at least about 60%, at least about 70%, or at least about 80%.
39. The method of any one of embodiments 1-38, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
40. The method of any one of embodiments 1-39, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
41. The method of any one of embodiments 1-40, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
42. The method of any one of embodiments 1-41, wherein the subject has one or more adverse events and is further administered an additional therapeutic agent to eliminate or reduce the severity of the one or more adverse events.
43. The method of any one of embodiments 1-41, wherein the subject is at risk of developing one or more adverse events and is further administered an additional therapeutic agent to prevent or reduce the severity of the one or more adverse events.
44. The method of embodiment 42 or embodiment 43, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.
45. The method of embodiment 42 or embodiment 43, wherein the one or more adverse events is a grade 3 or greater adverse event.
46. The method of embodiment 42 or embodiment 43, wherein the one or more adverse events is a serious adverse event.
47. The method of embodiment 42 or embodiment 43, wherein the one or more adverse events is conjunctivitis and/or keratitis and the additional agent is a preservative-free lubricating eye drop, an ocular vasoconstrictor and/or a steroid eye drop.
48. The method of any one of embodiments 1-47, wherein the antibody-drug conjugate is administered as a monotherapy.
49. The method of any one of embodiments 1-48, wherein the subject is a human.
50. The method of any one of embodiments 1-49, wherein the antibody-drug conjugate is in a pharmaceutical composition comprising the antibody-drug conjugate and a pharmaceutical acceptable carrier.
51. An antibody-drug conjugate that binds to tissue factor (TF) for use in a method of treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered to the subject at a dose ranging from about 1.5 mg/kg to about 2.1 mg/kg.
52. The antibody-drug conjugate for use of embodiment 51, wherein the dose is about 2.0 mg/kg.
53. The antibody-drug conjugate for use of embodiment 51 or embodiment 52, wherein the antibody-drug conjugate is administered once about every 1 week, 2 weeks, 3 weeks or 4 weeks.
54. The antibody-drug conjugate for use of any one of embodiments 51-53, wherein the antibody-drug conjugate is administered once about every 3 weeks.
55. The antibody-drug conjugate for use of any one of embodiments 51-54, wherein the subject has been previously treated with one or more therapeutic agents and did not respond to the treatment, wherein the one or more therapeutic agents is not the antibody-drug conjugate.
56. The antibody-drug conjugate for use of any one of embodiments 51-54, wherein the subject has been previously treated with one or more therapeutic agents and relapsed after the treatment, wherein the one or more therapeutic agents is not the antibody-drug conjugate.
57. The antibody-drug conjugate for use of any one of embodiments 51-54, wherein the subject has been previously treated with one or more therapeutic agents and has experienced disease progression during the treatment, wherein the one or more therapeutic agents is not the antibody-drug conjugate.
58. The antibody-drug conjugate for use of any one of embodiments 55-57, wherein the one or more therapeutic agents comprises a platinum-based therapeutic agent.
59. The antibody-drug conjugate for use of any one of embodiments 55-57, wherein the one or more therapeutic agents is selected from the group consisting of: paclitaxel, cisplatin, carboplatin, topotecan, gemcitabine, fluorouracil, ixabepilone, imatinib mesylate, docetaxel, gefitinib, paclitaxel, pemetrexed, vinorelbine, doxil, cetuximab, pembrolizumab, nivolumab and bevacizumab.
60. The antibody-drug conjugate for use of any one of embodiments 51-59, wherein the subject has experienced disease progression during or after treatment with:

a) paclitaxel and cisplatin,

b) paclitaxel and carboplatin, or

c) paclitaxel and topotecan.

61. The antibody-drug conjugate for use of any one of embodiments 51-60, wherein the subject has received treatment with bevacizumab.
62. The antibody-drug conjugate for use of any one of embodiments 51-60, wherein the subject is ineligible for treatment with bevacizumab.
63. The antibody-drug conjugate for use of any one of embodiments 51-62, wherein the subject is not a candidate for curative therapy.
64. The antibody-drug conjugate for use of embodiment 63, wherein the curative therapy comprises radiotherapy and/or exenterative surgery.
65. The antibody-drug conjugate for use of any one of embodiments 51-64, wherein the subject did not respond to treatment with no more than two prior systemic treatment regimens.
66. The antibody-drug conjugate for use of any one of embodiments 51-64, wherein the subject relapsed after treatment with no more than two prior systemic treatment regimens.
67. The antibody-drug conjugate for use of any one of embodiments 51-66, wherein the cervical cancer is an adenocarcinoma, an adenosquamous carcinoma or a squamous cell carcinoma.
68. The antibody-drug conjugate for use of any one of embodiments 51-67, wherein the cervical cancer is an advanced stage cervical cancer, such as a stage 3 or stage 4 cervical cancer, such as metastatic cervical cancer.
69. The antibody-drug conjugate for use of any one of embodiments 51-68, wherein the cervical cancer is recurrent cervical cancer.
70. The antibody-drug conjugate for use of any one of embodiments 51-69, wherein the monomethyl auristatin is monomethyl auristatin E (MMAE).
71. The antibody-drug conjugate for use of any one of embodiments 51-70, wherein the anti-TF antibody or antigen-binding fragment thereof of the antibody-drug conjugate is a monoclonal antibody or a monoclonal antigen-binding fragment thereof.
72. The antibody-drug conjugate for use of any one of embodiments 51-71, wherein the anti-TF antibody or antigen-binding fragment thereof of the antibody-drug conjugate comprises a heavy chain variable region and a light chain variable region, wherein the heavy chain variable region comprises:

(i) a CDR-H1 comprising the amino acid sequence of SEQ ID NO:1;

(ii) a CDR-H2 comprising the amino acid sequence of SEQ ID NO:2; and

(iii) a CDR-H3 comprising the amino acid sequence of SEQ ID NO:3; and

wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof of the antibody-drug conjugate are defined by the IMGT numbering scheme.

73. The antibody-drug conjugate for use of any one of embodiments 51-72, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
74. The antibody-drug conjugate for use of any one of embodiments 51-73, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
75. The antibody-drug conjugate for use of any one of embodiments 51-74, wherein the anti-TF antibody of the antibody-drug conjugate is tisotumab.
76. The antibody-drug conjugate for use of any one of embodiments 51-75, wherein the antibody-drug conjugate further comprises a linker between the anti-TF antibody or antigen-binding fragment thereof and the monomethyl auristatin.
77. The antibody-drug conjugate for use of embodiment 76, wherein the linker is a cleavable peptide linker.
78. The antibody-drug conjugate for use of embodiment 77, wherein the cleavable peptide linker has a formula: -MC-vc-PAB-, wherein:

### a) MC is:

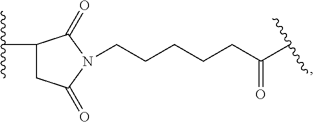 [SMILES: *C(=O)CCCCCN1C(=O)CC(*)C1=O]

b) vc is the dipeptide valine-citrulline, and

### c) PAB is:

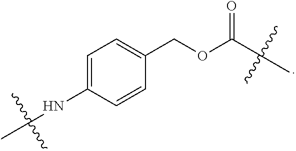 [SMILES: *Nc1ccc(COC(*)=O)cc1]

79. The antibody-drug conjugate for use of any one of embodiments 76-78, wherein the linker is attached to sulphydryl residues of the anti-TF antibody obtained by partial reduction or full reduction of the anti-TF antibody or antigen-binding fragment thereof.
80. The antibody-drug conjugate for use of embodiment 79, wherein the linker is attached to MMAE, wherein the antibody-drug conjugate has the following structure:

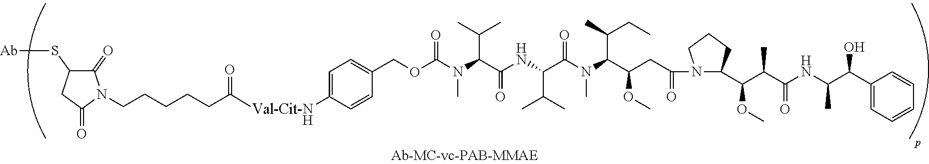

wherein p denotes a number from 1 to 8, S represents a sulphydryl residue of the anti-TF antibody, and Ab designates the anti-TF antibody or antigen-binding fragment thereof.
81. The antibody-drug conjugate for use of embodiment 80, wherein the average value of p in a population of the antibody-drug conjugates is about 4.
82. The antibody-drug conjugate for use of any one of embodiments 51-81, wherein the antibody-drug conjugate is tisotumab vedotin.
83. The antibody-drug conjugate for use of any one of embodiments 51-82, wherein the route of administration for the antibody-drug conjugate is intravenous.
84. The antibody-drug conjugate for use of any one of embodiments 51-83, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF.
85. The antibody-drug conjugate for use of any one of embodiments 51-84, wherein one or more therapeutic effects in the subject is improved after administration of the antibody-drug conjugate relative to a baseline.
86. The antibody-drug conjugate for use of embodiment 85, wherein the one or more therapeutic effects is selected from the group consisting of: size of a tumor derived from the cervical cancer, objective response rate, duration of response, time to response, progression free survival, and overall survival.
87. The antibody-drug conjugate for use of any one of embodiments 51-86, wherein the size of a tumor derived from the cervical cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cervical cancer before administration of the antibody-drug conjugate.
88. The antibody-drug conjugate for use of any one of embodiments 51-87, wherein the objective response rate is at least about 20%, at least about 25%, at least about 30%, at least about 35%, at least about 40%, at least about 45%, at least about 50%, at least about 60%, at least about 70%, or at least about 80%.
89. The antibody-drug conjugate for use of any one of embodiments 51-88, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
90. The antibody-drug conjugate for use of any one of embodiments 51-89, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
91. The antibody-drug conjugate for use of any one of embodiments 51-90, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
92. The antibody-drug conjugate for use of any one of embodiments 51-91, wherein the subject has one or more adverse events and is further administered an additional therapeutic agent to eliminate or reduce the severity of the one or more adverse events.
93. The antibody-drug conjugate for use of any one of embodiments 51-91, wherein the subject is at risk of developing one or more adverse events and is further administered an additional therapeutic agent to prevent or reduce the severity of the one or more adverse events.
94. The antibody-drug conjugate for use of embodiment 92 or embodiment 93, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.
95. The antibody-drug conjugate for use of embodiment 92 or embodiment 93, wherein the one or more adverse events is a grade 3 or greater adverse event.
96. The antibody-drug conjugate for use of embodiment 92 or embodiment 93, wherein the one or more adverse events is a serious adverse event.
97. The antibody-drug conjugate for use of embodiment 92 or embodiment 93, wherein the one or more adverse events is conjunctivitis and/or keratitis and the additional agent is a preservative-free lubricating eye drop, an ocular vasoconstrictor and/or a steroid eye drop.
98. The antibody-drug conjugate for use of any one of embodiments 51-97, wherein the antibody-drug conjugate is administered as a monotherapy.
99. The antibody-drug-conjugate for use of any one of embodiments 51-98, wherein the subject is a human.
100. The antibody-drug conjugate for use of any one of embodiments 51-99, wherein the antibody-drug conjugate is in a pharmaceutical composition comprising the antibody-drug conjugate and a pharmaceutical acceptable carrier.
101. Use of an antibody-drug conjugate that binds to tissue factor (TF) for the manufacture of a medicament for treating cervical cancer in a subject, wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered to the subject at a dose ranging from about 1.5 mg/kg to about 2.1 mg/kg.
102. The use of embodiment 101, wherein the dose is about 2.0 mg/kg.
103. The use of embodiment 101 or embodiment 102, wherein the antibody-drug conjugate is administered once about every 1 week, 2 weeks, 3 weeks or 4 weeks.
104. The use of any one of embodiments 101-103, wherein the antibody-drug conjugate is administered once about every 3 weeks.
105. The use of any one of embodiments 101-104, wherein the subject has been previously treated with one or more therapeutic agents and did not respond to the treatment, wherein the one or more therapeutic agents is not the antibody-drug conjugate.
106. The use of any one of embodiments 101-104, wherein the subject has been previously treated with one or more therapeutic agents and relapsed after the treatment, wherein the one or more therapeutic agents is not the antibody-drug conjugate.
107. The use of any one of embodiments 101-104, wherein the subject has been previously treated with one or more therapeutic agents and has experienced disease progression during the treatment, wherein the one or more therapeutic agents is not the antibody-drug conjugate.
108. The use of any one of embodiments 105-107, wherein the one or more therapeutic agents comprises a platinum-based therapeutic agent.
109. The use of any one of embodiments 105-107, wherein the one or more therapeutic agents is selected from the group consisting of: paclitaxel, cisplatin, carboplatin, topotecan, gemcitabine, fluorouracil, ixabepilone, imatinib mesylate, docetaxel, gefitinib, paclitaxel, pemetrexed, vinorelbine, doxil, cetuximab, pembrolizumab, nivolumab and bevacizumab.
110. The use of any one of embodiments 101-109, wherein the subject has experienced disease progression during or after treatment with:

a) paclitaxel and cisplatin,

b) paclitaxel and carboplatin, or

c) paclitaxel and topotecan.

111. The use of any one of embodiments 101-110, wherein the subject has received treatment with bevacizumab.
112. The use of any one of embodiments 101-110, wherein the subject is ineligible for treatment with bevacizumab.
113. The use of any one of embodiments 101-112, wherein the subject is not a candidate for curative therapy.
114. The use of embodiment 113, wherein the curative therapy comprises radiotherapy and/or exenterative surgery.
115. The use of any one of embodiments 101-114, wherein the subject did not respond to treatment with no more than two prior systemic treatment regimens.
116. The use of any one of embodiments 101-114, wherein the subject relapsed after treatment with no more than two prior systemic treatment regimens.
117. The use of any one of embodiments 101-116, wherein the cervical cancer is an adenocarcinoma, an adenosquamous carcinoma or a squamous cell carcinoma.
118. The use of any one of embodiments 101-117, wherein the cervical cancer is an advanced stage cervical cancer, such as a stage 3 or stage 4 cervical cancer, such as metastatic cervical cancer.
119. The use of any one of embodiments 101-118, wherein the cervical cancer is recurrent cervical cancer.
120. The use of any one of embodiments 101-119, wherein the monomethyl auristatin is monomethyl auristatin E (MMAE).
121. The use of any one of embodiments 101-120, wherein the anti-TF antibody or antigen-binding fragment thereof of the antibody-drug conjugate is a monoclonal antibody or a monoclonal antigen-binding fragment thereof.
122. The use of any one of embodiments 101-121, wherein the anti-TF antibody or antigen-binding fragment thereof of the antibody-drug conjugate comprises a heavy chain variable region and a light chain variable region, wherein the heavy chain variable region comprises:

(i) a CDR-H1 comprising the amino acid sequence of SEQ ID NO:1;

(ii) a CDR-H2 comprising the amino acid sequence of SEQ ID NO:2; and

(iii) a CDR-H3 comprising the amino acid sequence of SEQ ID NO:3; and

wherein the light chain variable region comprises:

(i) a CDR-L1 comprising the amino acid sequence of SEQ ID NO:4;

(ii) a CDR-L2 comprising the amino acid sequence of SEQ ID NO:5; and

(iii) a CDR-L3 comprising the amino acid sequence of SEQ ID NO:6, wherein the CDRs of the anti-TF antibody or antigen-binding fragment thereof of the antibody-drug conjugate are defined by the IMGT numbering scheme.

123. The use of any one of embodiments 101-122, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.
124. The use of any one of embodiments 101-123, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.
125. The use of any one of embodiments 101-124, wherein the anti-TF antibody of the antibody-drug conjugate is tisotumab.
126. The use of any one of embodiments 101-125, wherein the antibody-drug conjugate further comprises a linker between the anti-TF antibody or antigen-binding fragment thereof and the monomethyl auristatin.
127. The use of embodiment 126, wherein the linker is a cleavable peptide linker.
128. The use of embodiment 127, wherein the cleavable peptide linker has a formula: -MC-vc-PAB-, wherein:

### a) MC is:

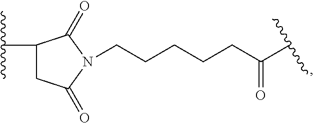 [SMILES: *C(=O)CCCCCN1C(=O)CC(*)C1=O]

b) vc is the dipeptide valine-citrulline, and

### c) PAB is:

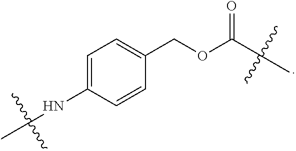 [SMILES: *Nc1ccc(COC(*)=O)cc1]

129. The use of any one of embodiments 126-128, wherein the linker is attached to sulphydryl residues of the anti-TF antibody obtained by partial reduction or full reduction of the anti-TF antibody or antigen-binding fragment thereof.
130. The use of embodiment 129, wherein the linker is attached to MMAE, wherein the antibody-drug conjugate has the following structure:

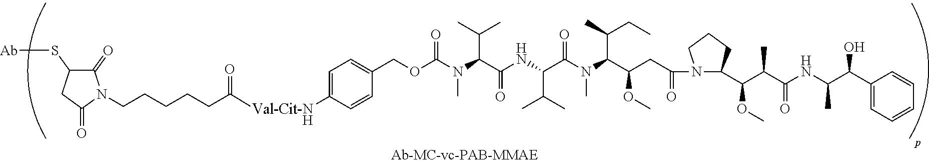

wherein p denotes a number from 1 to 8, S represents a sulphydryl residue of the anti-TF antibody, and Ab designates the anti-TF antibody or antigen-binding fragment thereof.
131. The use of embodiment 130, wherein the average value of p in a population of the antibody-drug conjugates is about 4.
132. The use of any one of embodiments 101-131, wherein the antibody-drug conjugate is tisotumab vedotin.
133. The use of any one of embodiments 101-132, wherein the route of administration for the antibody-drug conjugate is intravenous.
134. The use of any one of embodiments 101-133, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF.
135. The use of any one of embodiments 101-134, wherein one or more therapeutic effects in the subject is improved after administration of the antibody-drug conjugate relative to a baseline.
136. The use of embodiment 135, wherein the one or more therapeutic effects is selected from the group consisting of: size of a tumor derived from the cervical cancer, objective response rate, duration of response, time to response, progression free survival, and overall survival.
137. The use of any one of embodiments 101-136, wherein the size of a tumor derived from the cervical cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cervical cancer before administration of the antibody-drug conjugate.
138. The use of any one of embodiments 101-137, wherein the objective response rate is at least about 20%, at least about 25%, at least about 30%, at least about 35%, at least about 40%, at least about 45%, at least about 50%, at least about 60%, at least about 70%, or at least about 80%.
139. The use of any one of embodiments 101-138, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
140. The use of any one of embodiments 101-139, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
141. The use of any one of embodiments 101-140, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.
142. The use of any one of embodiments 101-141, wherein the subject has one or more adverse events and is further administered an additional therapeutic agent to eliminate or reduce the severity of the one or more adverse events.
143. The use of any one of embodiments 101-141, wherein the subject is at risk of developing one or more adverse events and is further administered an additional therapeutic agent to prevent or reduce the severity of the one or more adverse events.
144. The use of embodiment 142 or embodiment 143, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.
145. The use of embodiment 142 or embodiment 143, wherein the one or more adverse events is a grade 3 or greater adverse event.
146. The use of embodiment 142 or embodiment 143, wherein the one or more adverse events is a serious adverse event.
147. The use of embodiment 142 or embodiment 143, wherein the one or more adverse events is conjunctivitis and/or keratitis and the additional agent is a preservative-free lubricating eye drop, an ocular vasoconstrictor and/or a steroid eye drop.
148. The use of any one of embodiments 101-147, wherein the antibody-drug conjugate is administered as a monotherapy.
149. The use of any one of embodiments 101-148, wherein the subject is a human.
150. The antibody-drug conjugate for use of any one of embodiments 101-149, wherein the antibody-drug conjugate is in a pharmaceutical composition comprising the antibody-drug conjugate and a pharmaceutical acceptable carrier.
151. An article of manufacture comprising:

a) a medicament comprising an antibody-drug conjugate, wherein the antibody drug-conjugate comprises an anti-TF antibody or an antigen-binding fragment thereof conjugated to a monomethyl auristatin or a functional analog thereof or a functional derivative thereof; and

b) a package insert comprising instructions for administration of the medicament comprising the antibody-drug conjugate in a method of treating cervical cancer in a subject according to any one of embodiments 1-50 or the antibody-drug conjugate for use according to any one of embodiments 51-100 in a method for treating cervical cancer in a subject.

152. The article of manufacture of embodiment 151, wherein the medicament comprising the antibody-drug conjugate is in a container selected from group consisting of: a vial, a syringe, and an infusion bag.
153. The article of manufacture of embodiment 152, wherein the container comprises the antibody-drug conjugate at a dosage amount from about 4 mg to about 500 mg.
154. The article of manufacture of embodiment 152, wherein the container comprises the antibody-drug conjugate at a dosage amount from about 20 mg to about 60 mg.
155. The article of manufacture of embodiment 152, wherein the container comprises the antibody-drug conjugate at a concentration from about 5 mg/mL to about 15 mg/mL.
156. The article of manufacture of any one of embodiments 151-154, wherein the medicament comprising the antibody-drug conjugate is a lyophilized powder.
157. The article of manufacture of embodiment 156, wherein the lyophilized powder is reconstituted with a suitable diluent resulting in a final concentration from about 5 mg/mL to about 15 mg/mL.
158. The article of manufacture of any one of embodiments 151-157, wherein the medicament comprising the antibody-drug conjugate is for administration by intravenous infusion or injection.
159. The article of manufacture of embodiment 158, wherein the medicament comprising the antibody-drug conjugate is for administration by intravenous infusion.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: A Phase I/H Safety Study of Tisotumab Vedotin in Subjects with Cancer

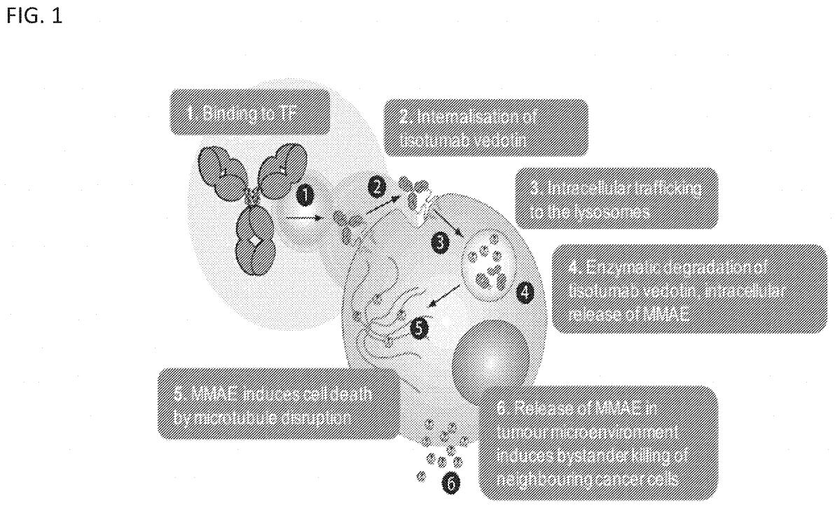

Tisotumab vedotin is an antibody-drug conjugate comprising an antibody that binds to tissue factor (TF), a protease-cleavable linker, and the microtubule disrupting agent MMAE. TF is a protein aberrantly expressed in a wide number of tumors including cervical cancer and is associated with poor prognosis. See Førster Y et al. Clin Chim Acta. 2006; 364 (1-2): 12-21 and Cocco E et al. BMC Cancer. 2011; 11:263. Tisotumab vedotin selectively targets TF to deliver a clinically validated toxic payload to tumor cells (FIG. 1). See Breij E C et al. Cancer Res. 2014; 74(4):1214-1226 and Chu A J. Int J Inflam. 2011; 2011. doi: 10.4061/2011/367284.

### Methods

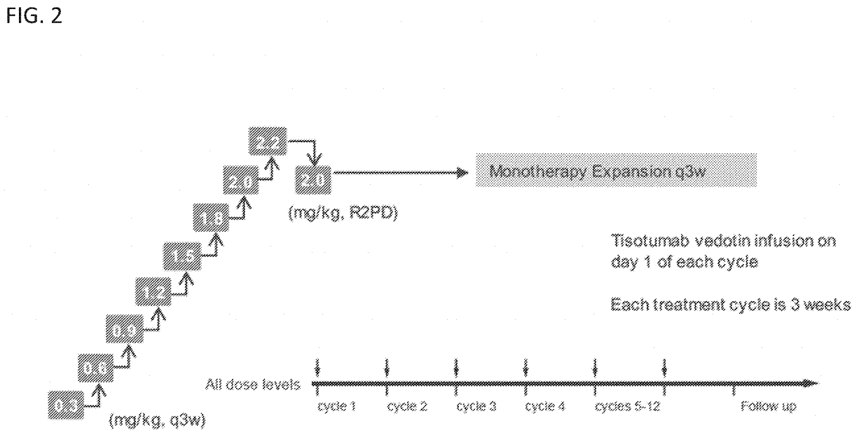

A first-in-human phase I/II dose-escalating study following a 3+3 dose-escalation design was conducted in order to test the safety of tisotumab vedotin in 27 subjects with locally advanced and/or metastatic cancer of various types including cervical cancer (FIG. 2). Tisotumab vedotin was administered by intravenous infusion at doses ranging from 0.3 mg/kg to 2.2 mg/kg on day 1 of a 21-day cycle for four cycles (i.e., each treatment cycle was 3 weeks). Patients with stable disease (SD) or better at the end of four cycles had the option to continue treatment with tisotumab vedotin for eight additional cycles (FIG. 2). Tumor evaluations were performed by CT scans every six weeks. In order to qualify for SD, results of the CT scan scheduled for week six needed to be SD or better. Two CT scans were performed outside the per-protocol defined window.

Lyophilized vials containing 40 mg of tisotumab vedotin were stored in a refrigerator at 2° C. to 8° C. Tisotumab vedotin was reconstituted in 4 ml of water leading to a reconstituted solution comprising 10 mg/mL tisotumab vedotin, 30 mM histidine, 88 mM sucrose, and 165 mM D-mannitol. The reconstituted antibody drug-conjugate solution had a pH of 6.0. The reconstituted tisotumab vedotin was diluted into a 0.9% NaCl 100 mL infusion bag according to the dose calculated for the patient. Intravenous infusion was completed within 24 hours after the tisotumab vedotin vial had been reconstituted. A 0.2 μm in-line filter was used for the intravenous infusion. The entire 100 mL volume from the prepared infusion bag was administered. No dead volume was provided.

A primary objective of the study was to assess the safety and tolerability in a mixed population of patients with specified solid tumors. Adverse event (AE) severity was graded according to Common Terminology Criteria for Adverse Events (CTCAE) version 4.03. Secondary objectives of the study included determining the pharmacokinetic (PK) profile of tisotumab vedotin and preliminary evaluation of anti-tumor activity as assessed according to Response Evaluation Criteria in Solid Tumors [RECIST] version 1.1. Dose-limiting toxicities (DLTs) were determined during the first cycle and were defined as grade ≥3 events possibly related to tisotumab vedotin. Maximum tolerated dose (MTD) was defined as the highest tisotumab vedotin dose level that did not cause unacceptable side effects. Tumor biopsies were required at baseline for TF expression which was assessed by immunohistochemistry utilizing tisotumab. TF staining intensity was determined using the H-scoring system.

Inclusion criteria for eligible subjects included: patients with relapsed, advanced, and/or metastatic cancer who have failed available standard therapy; and measurable disease.

Exclusion criteria included known past or current coagulation defects; ongoing major bleeding; and presence of CTCAE grade ≥2 peripheral neuropathy.

### Results

Patient demographics and baseline characteristics are shown in Table 1. A total of 25 patients withdrew from treatment due to patient choice (4%), disease progression (67%), dose-limiting toxicities (DLTs) (4%), AEs (15%), or death (4%). Two patients continued therapy beyond four cycles.

TABLE 1
Patient Demographics and Baseline Characteristics

| | | All |
|---|---|---|
| | | Patients |
| Patient Characteristics | | (n = 27) |
| Age in years, median (range) | | 62 (43-73) |
| Gender, number | Male | 9 (33%) |
| (% of total patients) | Female | 18 (67%) |
| Eastern Cooperative Oncology | 0 | 13 (48%) |
| Group (ECOG) performance | 1 | 13 (48%) |
| status (PS), number | NA | 1 (4%) |
| (% of total patients) | | |
| Primary tumor type, number | Cervix | 2 (7%) |
| (% of total patients) | Other tumor | 25 (93%) |
| | types | |

| | |
|---|---|
| Median number of prior | 3 (1-14) |
| therapies (range) | |

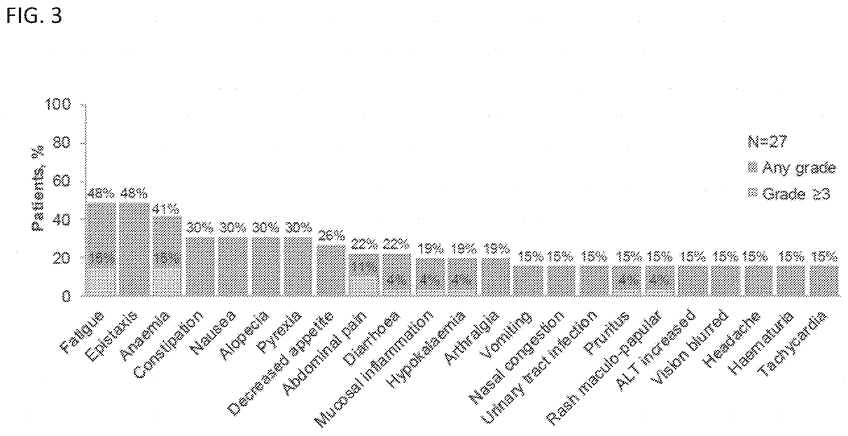

Safety analysis for all 27 patients is provided in Table 2. A total of 25 patients (93%) experienced treatment-related AEs, the most common of which were fatigue (48%), epitaxis (48%), and anaemia (41%). 19 patients experienced grade ≥3 treatment-related AEs, the most common of which were fatigue (n=4), anemia (n=4), abdominal pain (n=3), and hyponatraemia (n=3) (Table 2; FIG. 3). There were no grade 4 events. Seven patients discontinued due to AEs, which included grade 1 pneumonitis (n=1), grade 3 events for Guillain-Barre syndrome (n=1), diabetes mellitus (n=1), fatigue (n=1) and abdominal pain (n=2), and one patient experienced grade 2 peripheral swelling and grade 3 pain in extremity. There were three deaths reported in this study. One patient in the 0.6 mg/kg cohort died from tumor-related bleeding. Two patients in the 0.3 mg/kg cohort died from disease progression, both deaths were considered not related to the study drug. No significant changes in coagulation parameters were observed. The mean prothrombin time at baseline was 11.5 seconds (n=18) and 11.7 seconds by the end of the study (n=17). The mean activated partial thromboplastin time at baseline was 28.2 seconds (n=25) and 27.1 seconds (n=23) by the end of the study. Three DLTs (i.e., diabetes mellitus type 2, mucositis, and neutropenic fever, all grade 3) were seen in three patients in the 2.2 mg/kg dose cohort.

TABLE 2
Overall safety profile of tisotumab vedotin by dose cohorts

| AE category, | All | | 0.3 | | 0.6 | | 0.9 | | 1.2 | | 1.5 | | 1.8 | | 2.0 | | 2.2 | |
|---|---|---|---|---|---|---|---|---|---|---|---|---|---|---|---|---|---|---|
| n (% of total | doses | | mg/kg | | mg/kg | | mg/kg | | mg/kg | | mg/kg | | mg/kg | | mg/kg | | mg/kg | |
| patients)* | (n = 27) | | (n = 3) | | (n = 3) | | (n = 3) | | (n = 3) | | (n = 3) | | (n = 3) | | (n = 3) | | (n = 6) | |
| AE | 27 | (100) | 3 | (100) | 3 | (100) | 3 | (100) | 3 | (100) | 3 | (100) | 3 | (100) | 3 | (100) | 6 | (100) |
| Serious AE | 15 | (56) | 2 | (67) | 1 | (33) | 0 | | 2 | (67) | 2 | (67) | 2 | (67) | 2 | (67) | 4 | (67) |
| Grade ≥3 AE | 19 | (70) | 2 | (67) | 3 | (100) | 2 | (67) | 1 | (33) | 2 | (67) | 3 | (100) | 2 | (67) | 4 | (67) |
| Treatment- | 25 | (93) | 3 | (100) | 3 | (100) | 1 | (33) | 3 | (100) | 3 | (100) | 3 | (100) | 3 | (100) | 6 | (100) |
| related AE | | | | | | | | | | | | | | | | | | |

| | | | | | | | | | | | | |
|---|---|---|---|---|---|---|---|---|---|---|---|---|
| AE leading to | 7 | (26) | 0 | 0 | 0 | 0 | 0 | 2 | (67) | 0 | 5 | (83) |
| discontinuation | | | | | | | | | | | | |

| | | | | | | | | | | | | |
|---|---|---|---|---|---|---|---|---|---|---|---|---|
| AE with | 3 | (11) | 2 | (67) | 1 | (33) | 0 | 0 | 0 | 0 | 0 | 0 |
| outcome of | | | | | | | | | | | | |
| death | | | | | | | | | | | | |

*Occurring up to 30 days after treatment. AE indicates adverse event.

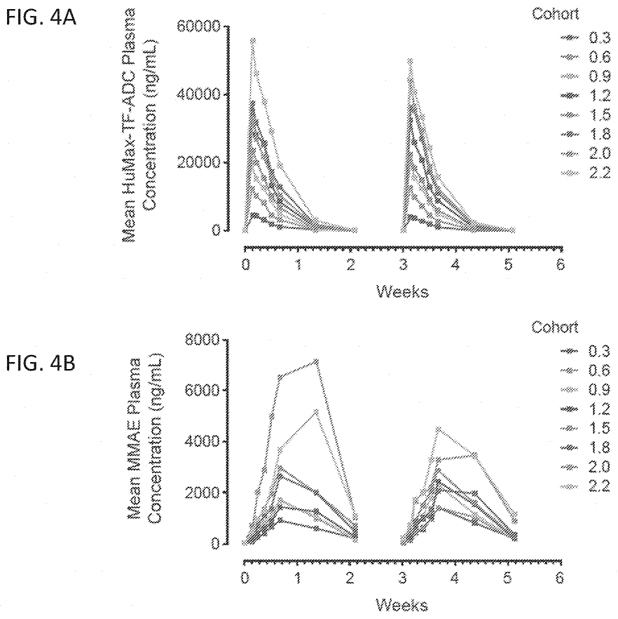

The geometric means (% CV) of the time to reach Cₘₐₓ (Tₘₐₓ) (hr), maximum concentration (Cₘₐₓ)(ng/mL), and area under the concentration time curve (AUC)₀₋ₜ (hr*ng/mL) was measured for the pharmacokinetics (PK) portion of the study (Table 3). Low levels of unconjugated MMAE were measured in systemic circulation (FIG. 4).

TABLE 3
Summary of tisotumab vedotin plasma
PK parameters by dose cohorts in cycle 1

| Dose | | Tₘₐₓ | Cₘₐₓ | AUC₀₋ₜ |
|---|---|---|---|---|
| (mg/kg) | N | (hr) | (ng/ml) | (hr*ng/ml) |
| 0.3 | 3 | 1.5 (73%) | 4782.7 (12%) | 59216.8 (3%) |
| 0.6 | 3 | 1.2 (13%) | 12195.3 (10%) | 368432.7 (8%) |
| 0.9 | 3 | 1.3 (12%) | 19811.6 (17%) | 601926.2 (17%) |
| 1.2 | 3 | 1.3 (12%) | 34673.1 (19%) | 1084672.7 (9%) |
| 1.5 | 3 | 1.1 (9.6%) | 23115.6 (21%) | 794988.4 (19%) |
| 1.8 | 3 | 1.2 (14%) | 35416.3 (39%) | 1504823.8 (50%) |
| 2.0 | 3 | 1.2 (8%) | 32296.1 (22%) | 1256379.7 (33%) |
| 2.2 | 6 | 1.1 (13%) | 55530.3 (10%) | 2037070.5 (34%) |

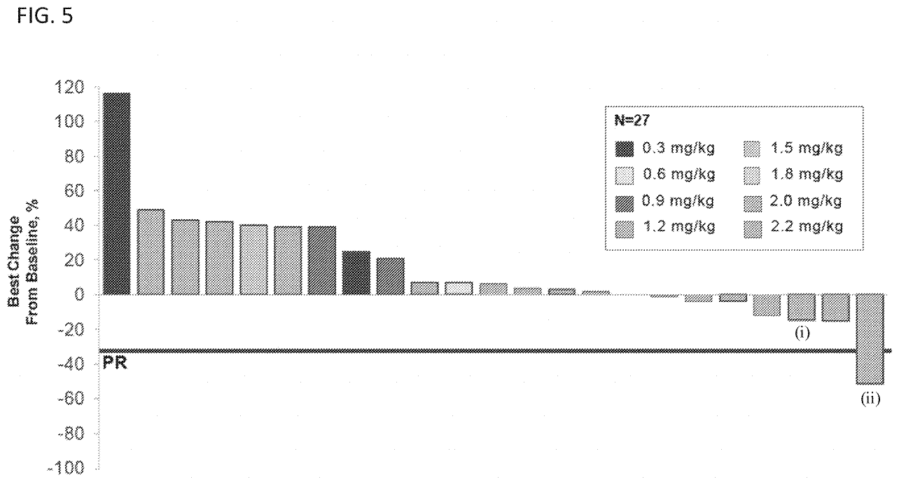

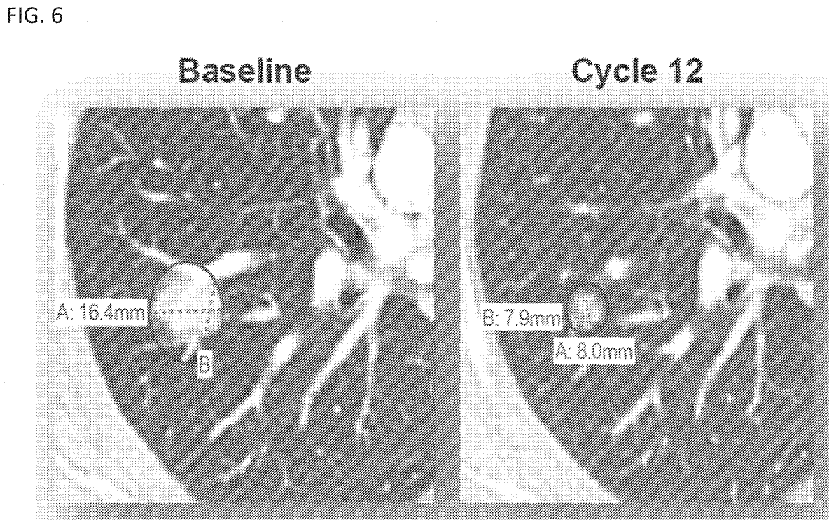

Twenty-six patients were evaluated for efficacy (Table 4). The best response observed was partial response (PR) in 1 patient (4%) and stable disease (SD) in 11 patients (41%). The disease control rate (DCR; PR+SD) was 46% corresponding to 12 out of 26 patients. Changes in tumor size, expressed as a percentage of baseline, in 27 patients was determined (FIG. 5). The tumor size in the two patients with cervical cancer was reduced relative to baseline with Patient 1 exhibiting about 20% reduction in tumor size when administered 2.2 mg/kg of tisotumab vedotin (FIG. 5; (i)) and Patient 2 exhibiting about 51% reduction in tumor size when administered 1.2 mg/kg of tisotumab vedotin (FIG. 5; (ii)). Patient 2 was a 43 year old cervical cancer patient diagnosed with stage 4 disease who had received 3 prior lines of therapy. TF expression in Patient 2 was measured at an H-score of 140 (archival). Patient 2 achieved a confirmed PR, with about 51% reduction in the target lesion and continued benefit for a total of 15 months (FIG. 6). Tisotumab vedotin was well tolerated and no severe AEs were reported. Patient 2 eventually experienced disease progression and stopped therapy.

TABLE 4
Confirmed objective responses

Confirmed
Response per

| RECIST v1.1, | All | | 0.3 | 0.6 | | 0.9 | | 1.2 | | 1.5 | 1.8 | 2.0 | | 2.2 | |
|---|---|---|---|---|---|---|---|---|---|---|---|---|---|---|---|
| n (% of total | doses | | mg/kg | mg/kg | | mg/kg | | mg/kg | | mg/kg | mg/kg | mg/kg | | mg/kg | |
| patients)^{a} | (n = 27) | | (n = 3) | (n = 3) | | (n = 3) | | (n = 3) | | (n = 3) | (n = 3) | (n = 3) | | (n = 6) | |
| Complete | 0 | | 0 | 0 | | 0 | | 0 | | 0 | 0 | 0 | | 0 | |
| response (CR) | | | | | | | | | | | | | | | |
| Partial | 1 | (4) | 0 | 0 | | 0 | | 1 | (33) | 0 | 0 | 0 | | 0 | |
| response (PR) | | | | | | | | | | | | | | | |
| Stable disease | 11 | (41) | 0 | 1 | (33) | 1 | (33) | 1 | (33) | 0 | 3 (100) | 1 | (33) | 4 | (67) |
| (SD) | | | | | | | | | | | | | | | |
| Progressive | 14 | (52) | 3 (100) | 1 | (33) | 2 | (67) | 1 | (33) | 3 (100) | 0 | 2 | (67) | 2 | (33) |
| disease (PD) | | | | | | | | | | | | | | | |

| | | | | | | | | | | | |
|---|---|---|---|---|---|---|---|---|---|---|---|
| Not evaluable^{b} | 1 | (4) | 0 | 1 | (33) | 0 | 0 | 0 | 0 | 0 | 0 |

^{a}Percentages may not add to 100% due to rounding.
^{b}Patient died prior to first scan.

### Conclusion

The MTD was identified as 2.0 mg/kg and used in a Phase II study on the efficacy and safety of tisotumab vedotin in patients with cervical cancer.

### Example 2: Effect of a Tisotumab Vedotin in a Phase IIa Study in Subjects with Relapsed, Recurrent and/or Metastatic Cervical Cancer

The efficacy, safety and tolerability of 2.0 mg/kg tisotumab vedotin in patients with relapsed, recurrent, and/or metastatic cervical cancer were evaluated.

### Methods

A Phase IIa single arm, multicenter trial investigated the efficacy, safety and tolerability of 2.0 mg/kg tisotumab vedotin in patients with relapsed, recurrent and/or metastatic cervical cancer. A total of 34 patients (n=34) were enrolled and received at least 1 dose of tisotumab vedotin. Each eligible patient was assigned to receive an intravenous (IV) infusion dose of tisotumab vedotin at a concentration of 2.0 mg/kg on day 1 of a 21-day cycle (i.e., each treatment cycle was 3 weeks (q3w)).

Lyophilized vials containing 40 mg of tisotumab vedotin were stored in a refrigerator at 2° C. to 8° C. Tisotumab vedotin was reconstituted in 4 ml of water leading to a reconstituted solution comprising 10 mg/mL tisotumab vedotin, 30 mM histidine, 88 mM sucrose, and 165 mM D-mannitol. The reconstituted antibody drug-conjugate solution had a pH of 6.0. The reconstituted tisotumab vedotin was diluted into a 0.9% NaCl 100 mL infusion bag according to the dose calculated for the patient to receive 2.0 mg/kg tisotumab vedotin. Intravenous infusion was completed within 24 hours after the tisotumab vedotin vial had been reconstituted. A 0.2 μm in-line filter was used for the intravenous infusion. The entire 100 mL volume from the prepared infusion bag was administered. No dead volume was provided.

A primary objective of the study was to assess safety and tolerability of tisotumab vedotin. Adverse event (AE) severity was graded according to CTCAE version 4.03. A secondary objective was preliminary evaluation of anti-tumor activity durability as assessed according to RECIST version 1.1. Tumor evaluations were performed by CT scans every six weeks.

### Results

Patient demographics and baseline characteristics are shown in Table 5. A total of 7 patients continued with therapy (22%) and 27 patients withdrew from treatment due to AE (n=5), disease progression (n=16), or other reasons (n=6).

TABLE 5
Patient Demographics and Baseline Characteristics

| | | All Patients |
|---|---|---|
| Patient Characteristics | | (n = 34) |
| Age in years, median (range) | | 43 (21-73) |
| ECOG score, number | 0 | 7 (21%) |
| (% of total patients) | 1 | 26 (76%) |
| | Missing | 1 (3%) |
| Cancer type, number | Adenocarcinoma | 15 (44%) |
| (% of total patients) | Adeno-squamous | 3 (9%) |
| | Squamous | 15 (44%) |
| | Missing | 1 (3%) |
| Previous lines of systemic | 0^{a} | 3 (9%) |
| treatments, number | 1 | 13 (38%) |
| (% of total patients) | 2 | 11 (32%) |
| | 3 | 4 (12%) |
| | 4 | 3 (9%) |
| Prior treatments, %^{b} | Platinum | 91% |
| | Taxane | 91% |
| | Bevacizumab ^{c} | 71% |
| | GOG 240 regimen ^{d} | 68% |
| | ≥1 platinum doublet | 17% |

| | |
|---|---|
| Prior radiotherapy ^{e} | 74% |

^{a}Patients progressed on therapy administered for treatment of locally advanced disease.
^{b}Missing data from one patient.
^{c} Including bevacizumab administered as combination therapy as either platinum/bevacizumab/paclitaxel or topotecan/bevacizumab/paclitaxel.
^{d} Combination therapy with cisplatin, paclitaxel and bevacizumab
^{e} External beam radiotherapy administered to the cervix or surrounding tissues.

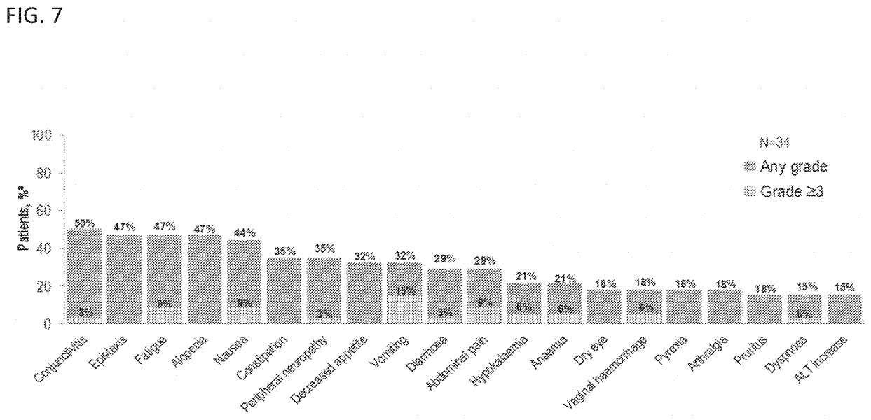

Common (≥15%) AEs following tisotumab vedotin monotherapy were evaluated (FIG. 7). Grade 3 AEs were reported in 16 patients (47%). There were no grade 4 or grade 5 events. Compound-specific conjunctival toxicity was observed, however mitigation measures substantially reduced conjunctival toxicity in patients. Prior to mitigation (n=15), 73% of patients experienced conjunctivitis of any grade. After mitigation (n=19), 32% of patients experienced conjunctivitis of any grade, and 5% at grade ≥3. Risk mitigation measures involved a prophylactic steroid, lubricating eye drops, and cooling eye masks worn during treatment by IV infusion, as well as stricter dose adjustment guidance.

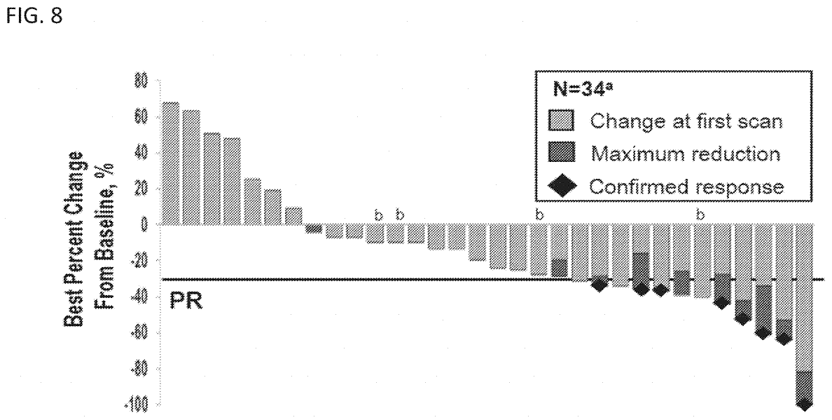

The thirty-four patients were evaluated for efficacy (Table 6 and FIG. 8). Seven patients continued to undergo treatment.

TABLE 6
Efficacy measurement

| Tumor response, PFS ^{a}, and DoR ^{b} | |
|---|---|
| n (% of total patients) | N = 34 |
| Objective response rate (ORR), n | 11 (32%) |
| (% of total patients) | (17%-50%) |
| (95% Confidence interval) | |
| Partial response (PR), n | 11 (32%) |
| (% of total patients)^{c} | |
| DCR (CR + PR + SD) ^{d}, n | 17 (50%) |
| (% of total patients) | (35%-65%) |
| (95% Confidence interval) | |
| Median DoR, months^{e} | 8.3 |
| Median PFS, months | 6.4 |

^{a} PFS indicates progression-free survival.
^{b} DoR indicates duration of response.
^{c}Including 8 confirmed PR and 3 unconfirmed PR (1 of which is still ongoing).
^{d} Clinical benefit after 12 weeks. DCR indicates disease control rate, CR indicates complete response, SD indicates stable disease.
^{e}Median DoR of 5.4 months for confirmed and unconfirmed responses.

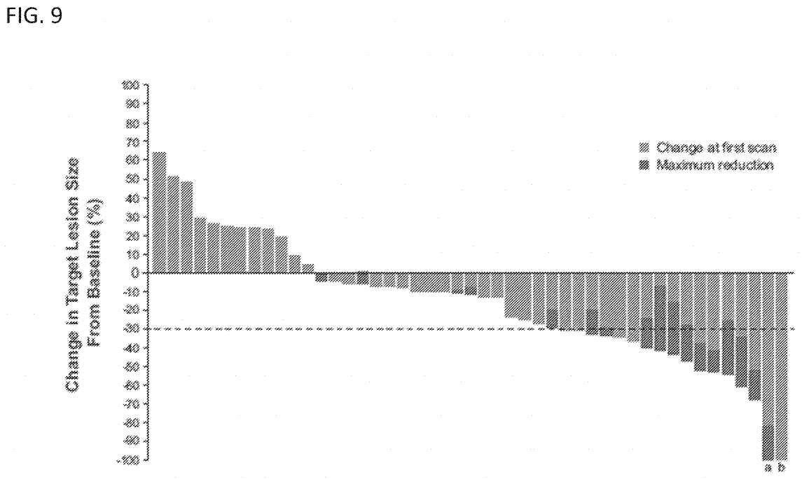

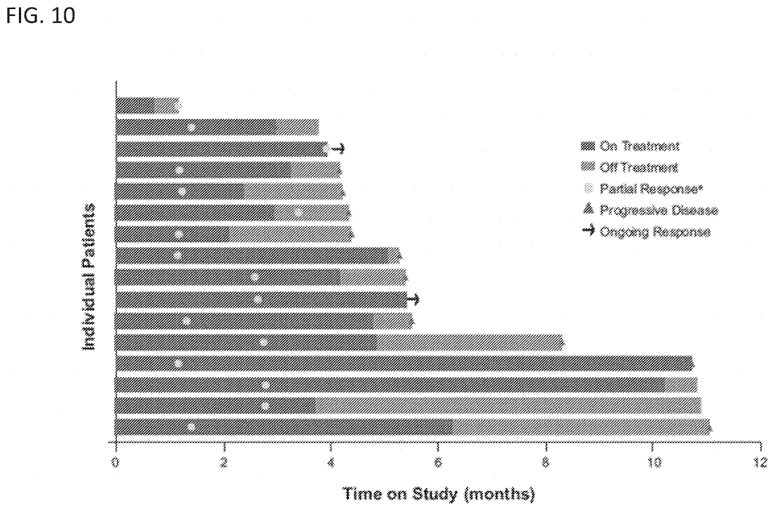

The trial was subsequently expanded to include additional patients. A total of 55 patients were evaluated for efficacy (Table 7, FIG. 9 and FIG. 10). Four patients continued to undergo treatment.

TABLE 7
Efficacy measurement

| | | ORR^{a}, | ORR 95% |
|---|---|---|---|
| | N | N (%)^{b} | CI, % |
| All efficacy-evaluable patients | 51 | 16 (31) | 19-46 |
| Histology | | | |
| Squamous | 27 | 9 (33) | 17-54 |
| Adenocarcinoma | 18 | 4 (22) | 6-48 |
| Adenosquamous | 4 | 2 (50) | 7-93 |
| Other | 2 | 1 (50) | 1-99 |
| Prior lines of systemic therapy | | | |
| 1 | 23 | 8 (35) | 16-57 |
| 2 | 17 | 6 (35) | 14-62 |
| 3-4 | 11 | 2 (18) | 2-52 |
| Prior taxane | | | |
| Yes | 48 | 15 (31) | 19-46 |
| No | 3 | 1 (33) | 1-91 |
| Prior bevacizumab | | | |
| Yes | 40 | 12 (30) | 17-47 |
| No | 11 | 4 (36) | 11-69 |
| Prior GOG 240 regimen^{c} | | | |
| Yes | 37 | 12 (32) | 18-50 |
| No | 14 | 4 (29) | 8-58 |

^{a}Indicates Objective Response Rate.
^{b}combined unconfirmed + confirmed ORR.
^{c}GOG 240 regimen defined as bevacizumab + doublet chemotherapy (cisplatin + paclitaxel or topotecan + paclitaxel.

### Conclusion

Tisotumab vedotin demonstrated robust efficacy and a manageable safety profile in the cervical cancer cohort. The safety profile of tisotumab vedotin in recurrent cervical cancer was generally consistent with other MMAE-based ADCs. Compound-specific conjunctival events were observed, however mitigation measures substantially reduced toxicity.

### Example 3: A Phase II Trial of Tisotumab Vedotin in Subjects with Previously Treated, Recurrent or Metastatic Cervical Cancer

The efficacy, safety and tolerability of 2.0 mg/kg tisotumab vedotin in patients with previously treated, advanced cervical cancer (e.g., recurrent and/or metastatic cancer) is evaluated. Preliminary data observed in a cohort of previously treated cervical cancer patients suggest a positive benefit risk profile for this population of high unmet need. See Example 1 and 2 above.

### Methods

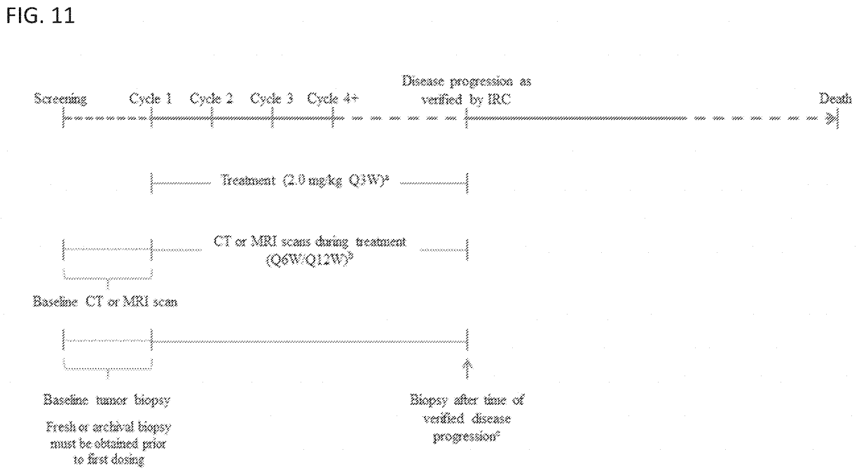

This phase II single arm, multicenter, international trial evaluates the efficacy, safety and tolerability of 2.0 mg/kg tisotumab vedotin in patients with recurrent or metastatic cervical cancer. Eligible patients have experienced disease progression during or after treatment with a chemotherapy doublet in combination with bevacizumab if eligible to receive bevacizumab. Patients have received no more than 2 prior systemic therapies for their metastatic or recurrent disease. Eligible patients are treated with intravenous (IV) tisotumab vedotin 2.0 mg/kg, every 3 weeks (1Q3W) until they meet a predefined discontinuation criterion (FIG. 11). Imaging is obtained every six weeks for the first 30 weeks and every 12 weeks thereafter. Responses are confirmed no earlier than 4 weeks (28 days) after the first assessment of response. Approximately 100 patients, age 18 years, are enrolled into the trial.

Inclusion criteria and exclusion criteria for patients enrolled in trial are shown in Table 8.

TABLE 8
List of inclusion and exclusion criteria

| | |
|---|---|
| Inclusion Criteria | Patients with extra-pelvic metastatic or recurrent cervical cancer |
| | including squamous cell, adeno carcinoma or adeno squamous histology, |
| | that: |
| | Have experienced disease progression during or after treatment with: |
| | chemotherapy doublet including paclitaxel and cisplatin |
| | or carboplatin |
| | OR |
| | paclitaxel and topotecan, |
| | and who have received or are ineligible for treatment with |
| | bevacizumab according to local standards. |
| | Have received no more than 2 prior systemic treatment |
| | regimens for recurrent or metastatic cervical cancer. |
| | Are not candidates for curative therapy, including but not |
| | limited to, radiotherapy or exenterative surgery. |
| | Measurable disease according to RECIST v1.1 as assessed by |
| | independent central imaging review. |
| | Age ≥18 years. |
| | Acceptable renal function: Calculated (Cockcroft-Gault) |
| | Glomerular Filtration Rate (GFR) >45 mL/min. |
| | Acceptable liver function: Alanine aminotransferase (ALT) and |
| | aspartate aminotransferase (AST) ≤3 times the upper limit of |
| | normal (ULN) (if liver tumor/metastases are present, then ≤5 × |
| | ULN is allowed); bilirubin ≤1.5 × ULN, except in patients |
| | diagnosed with Gilbert's syndrome, direct bilirubin ≤2 × ULN. |
| | Acceptable hematological status: Hemoglobin ≥5.6 mmol/L |
| | (9.0 g/dL), absolute neutrophil count (ANC) ≥1500/μL |
| | (1.5 × 109/L); platelet count ≥100 × 109/L assessed at least 2 |
| | weeks after transfusion with blood products and/or growth factor |
| | support. |
| | Acceptable coagulation status: International normalized ratio |
| | (INR) ≤1.2 (patients not on anti-coagulation therapy), and |
| | activated partial thromboplastin time (aPTT) ≤1.25 ULN; |
| | patients on anti-coagulation therapy (e.g., warfarin) must be on a |
| | steady dose (no active titration) for at least 4 weeks prior to |
| | screening and must have an INR ≤2.5 for eligibility. |
| | Eastern Cooperative Oncology Group (ECOG) performance |
| | status of 0 or 1 assessed within 7 days of cycle 1 day 1. |
| | Life expectancy of at least three months. |
| | A negative serum pregnancy test (in patients 18-55 years of age; |
| | post-menopause must be confirmed in eCRF for patients >55 |
| | years). Women who are pregnant or breast feeding are ineligible. |
| | Patients of reproductive potential must agree to use adequate |
| | contraception during and for 6 months after the last |
| | administration of tisotumab vedotin. Adequate contraception for |
| | women is defined as highly effective methods of contraception. |
| | In countries where two highly effective methods of |
| | contraception are required this will be an inclusion criterion. |
| | All patients must provide biopsy specimen during screening. |
| | Archival or fresh core biopsies are required (aspirates are not |
| | acceptable). FFPE blocks OR at least 10 slides with 5 micron |
| | thick sections are acceptable for eligibility. |
| | Following receipt of verbal and written information about the |
| | trial, patients must provide signed informed consent before any |
| | trial-related activity is carried out. |
| Exclusion Criteria | Hematological: Known past or current coagulation defects |
| | leading to an increased risk of bleeding; diffuse alveolar |
| | hemorrhage from vasculitis; known bleeding diathesis; ongoing |
| | major bleeding; trauma with increased risk of life-threatening |
| | bleeding or history of severe head trauma or intracranial surgery |
| | within two months of trial entry. |
| | Cardiovascular: Clinically significant cardiac disease including |
| | unstable angina, acute myocardial infarction 6 months prior to |
| | screening; known congestive heart failure (Grade III or IV as |
| | classified by the New York Heart Association), and/or a known |
| | decreased cardiac ejection fraction of <45%; a marked baseline |
| | prolongation of QT/QTc interval (e.g., repeated demonstration |
| | of a QTc interval >450 msec), a complete left bundle branch |
| | block (defined as a QRS interval ≥120 msec in left bundle |
| | branch block form) or an incomplete left bundle branch block. |
| | Central nervous system: Any history of intracerebral |
| | arteriovenous malformation, cerebral aneurysm, or stroke |
| | (transient ischemic attack >1 month prior to screening is |
| | allowed). |
| | Ophthalmological: Active ocular surface disease at baseline (as |
| | evaluated by ophthalmologist in case active ocular surface |
| | disease is suspected by the investigator). Patients with any prior |
| | episode of cicatricial conjunctivitis or Steven Johnson syndrome |
| | (as evaluated by the investigator) are ineligible. |
| | Other cancer/metastases: Known past or current malignancy |
| | other than inclusion diagnosis, except for: non-invasive basal |
| | cell or squamous cell skin carcinoma; noninvasive, superficial |
| | bladder cancer; any curable cancer with a complete response |
| | (CR) of >5 years duration. |
| | Brain metastases are allowed if the following criteria are |
| | met: Definitive therapy (for example: surgery or stereotactic |
| | brain radiotherapy) has been completed >28 days before the |
| | first dose of tisotumab vedotin ; the patient has no evidence |
| | of clinical or radiologic tumor progression; patients have |
| | completed perioperative corticosteroid therapy or steroid |
| | taper. Chronic steroid therapy is acceptable provided that the |
| | dose is stable for 1 month prior to screening. |
| | Excluded medications or treatment regimens: Therapeutic anti- |
| | coagulation therapy or anti-platelet therapy UNLESS the patient |
| | is no longer being actively titrated for their anti-coagulation (e.g. |
| | warfarin) and is on steady doses for at least 4 weeks prior to |
| | screening; cumulative dose of corticosteroid ≥150 mg |
| | (prednisone or equivalent doses of corticosteroids) within 2 |
| | weeks of the first tisotumab vedotin administration. |
| | Surgery/procedures: Major surgery within 4 weeks or open |
| | biopsy within 7 days prior to the first tisotumab vedotin |
| | administration. Patients who have planned major surgery during |
| | the treatment period must be excluded from the trial. |
| | Peripheral neuropathy grade ≥2 |
| | Prior therapy: |
| | Any prior treatment with MMAE-derived drugs. |
| | Any anti-cancer therapy, including small molecules, |
| | immunotherapy, chemotherapy, monoclonal antibodies, or |
| | any other experimental drug within 28 days prior to first |
| | tisotumab vedotin administration. Patients, who have not |
| | recovered from symptomatic side effects of radiotherapy or |
| | symptoms of autoimmune toxicities related to prior immune |
| | therapy at the time of initiation of screening procedure, are |
| | not eligible. |
| | Other: Ongoing significant, uncontrolled medical condition; |
| | clinically significant active viral, bacterial or fungal infection |
| | requiring IV or oral (PO) treatment with antimicrobial therapy |
| | ending less than 7 days prior to first tisotumab vedotin |
| | administration; known human immunodeficiency virus |
| | seropositivity; known history of hepatitis B or C infection. |
| | Patient has any condition for which, in the opinion of the |
| | investigator, participation would not be in the best interest of the |
| | patient (e.g. compromise the well-being) or that could prevent, |
| | limit, or confound the protocol-specified assessments. |
| | Patient has known allergies, hypersensitivity, or intolerance to |
| | tisotumab vedotin or its excipients. |

Lyophilized vials containing 40 mg of tisotumab vedotin are stored in a refrigerator at 2° C. to 8° C. Tisotumab vedotin is reconstituted in 4 ml of water leading to a reconstituted solution comprising 10 mg/mL tisotumab vedotin, 30 mM histidine, 88 mM sucrose, and 165 mM D-mannitol. The reconstituted antibody drug-conjugate solution has a pH of 6.0. The reconstituted tisotumab vedotin is diluted into a 0.9% NaCl 100 mL infusion bag according to the dose calculated for the patient to receive 2.0 mg/kg tisotumab vedotin. Intravenous infusion is completed within 24 hours after the tisotumab vedotin vial has been reconstituted. A 0.2 μm in-line filter is used for the intravenous infusion. The entire 100 mL volume from the prepared infusion bag is administered. No dead volume is provided. For patients that do not tolerate the protocol-specified dosing schedule, dose reductions are permitted in order to allow the patient to continue treatment with tisotumab vedotin (Table 9).

TABLE 9
Dose Modification Scheme

| Previous dose of | Reduced dose of |
|---|---|
| tisotumab vedotin | tisotumab vedotin |
| 2.0 mg/kg | 1.3 mg/kg |
| 1.3 mg/kg | 0.9 mg/kg |
| 0.9 mg/kg | 0.9 mg/kg* |

*If the patient is already being treated with tisotumab vedotin 0.9 mg/kg 1Q3W, the dose of tisotumab vedotin is not reduced further.

Objectives and endpoints are described in Table 10. The confirmed objective response rate (ORR) and a 2-sided 95% exact confidence interval is calculated 27 weeks after the last patient has received the first dose of tisotumab vedotin. Assuming a true confirmed ORR of 25% for tisotumab vedotin, 100 patients provides 96% power to exclude an ORR of 11% or less (one-sided P-value of 2.5%).

TABLE 10
Objectives and endpoints

| | |
|---|---|
| OBJECTIVES | ENDPOINTS |

Primary

| | |
|---|---|
| Determine the anti-tumor | Confirmed objective response |
| efficacy in patients with | rate (ORR) based upon |
| cervical cancer. | RECIST v1.1 assessed by the |
| | independent review committee. |

Secondary

| | |
|---|---|
| Evaluate durability and | Duration of response (DOR). |
| time to response. | Time to response (TTR). |
| Evaluate other clinical | Confirmed ORR by RECIST |
| outcomes. | v1.1, investigator assessment. |
| Assess safety and | Progression free survival (PFS) |
| tolerability. | by RECIST v1.1 by IRC. |
| | Overall survival (OS). |
| | Adverse events and safety |
| | laboratory parameters. |
| | Pharmacokinetics (PK). |
| | Immunogenicity (Anti- |
| | Drug Antibodies [ADAs]) |
| | of tisotumab vedotin. |

Exploratory

| | |
|---|---|
| Assess biomarkers related | TF expression in pre-treatment |
| to clinical response. | and post-progression tumor |
| Assess potential | biopsies, circulating TF, |
| pharmacodynamic | proteomic analyses and |
| biomarkers of tisotumab | genetic variations. |
| vedotin. | Circulating TF and proteomic |
| Assess Health Related | analyses. |
| Quality of Life (HRQL) | HRQL relevant questionnaires. |
| in cervical cancer | |
| patients treated with | |
| tisotumab vedotin. | |

If a patient's trial treatment is discontinued before the end of the treatment regimen, this does not result in automatic withdrawal of the patient from the trial. A patient's trial treatment is discontinued if: radiographic disease progression is verified by independent committee review; safety stopping rules are fulfilled; unacceptable toxicity requires treatment discontinuation; the investigator believes that for safety reasons (e.g., adverse event) it is in the best interest of the patient to stop treatment; pregnancy; patient choice; and/or a new anti-cancer therapy is initiated. When treatment is discontinued, investigators perform a safety follow-up visit. The safety follow-up visit is performed 15 days±5 days after the last dose of tisotumab vedotin and prior to initiation of new anti-cancer treatment and includes most assessments performed at screening and response assessments. Upon treatment discontinuation, patients continue to be followed for post-treatment assessments until death or withdrawal from the trial. Safety stopping rules for discontinuation of treatment include the following in case of ocular toxicity: first recurrence of CTCAE grade ≥3 conjunctivitis (despite dose reduction); third recurrence of CTCAE grade ≤2 keratitis (despite dose reductions); first occurrence of CTCAE grade ≥3 keratitis; ophthalmological evaluation reveals conjunctival/corneal scarring; any grade of symblepharon; any grade of fluorescent patches or conjunctival ulceration that does not stabilize or improve after dose reduction; or any dose delay related to ocular toxicity exceeding 12 weeks. Safety stopping rules for discontinuation of treatment include the following in case of other adverse events besides ocular toxicity: second occurrence of a grade 3 infusion related reaction (despite pre-medication); first occurrence of a ≥grade 4 infusion related reaction; first occurrence of mucositis ≥grade 4; first occurrence of peripheral neuropathy ≥grade 4; any event of pulmonary or CNS hemorrhage ≥grade 2; or any event of hemorrhage ≥grade 3 for patients on anti-coagulation therapy.

Three adverse events of special interest are ocular adverse events, adverse events of peripheral neuropathy, and adverse events of bleeding. For ocular AEs: AEs of grade 1-2 conjunctivitis are frequently reported in relation to treatment with tisotumab vedotin. Severe cases (CTCAE ≥grade 3) of conjunctivitis and keratitis are observed, however implementation of a comprehensive mitigation plan and preventive measures substantially reduce both the frequency and severity of ocular adverse reactions. In order to prevent ocular AEs, the following ocular pre-medication guidelines are followed: use of preservative-free lubricating eye drops from the start of treatment with tisotumab vedotin until the end of treatment; avoid use of contact lenses while treated with tisotumab vedotin; use of refrigerator-based eye cooling pads during infusion, e.g. THERA PEARL Eye Mask or similar, to be applied immediately before infusion in accordance with the instructions provided with the eye cooling pads; administration of local ocular vasoconstrictor before infusion (brimonidine tartrate 0.2% eye drops or similar, 3 drops in each eye immediately prior to start of infusion; otherwise to be used in accordance with the product prescribing information). If the patient does not tolerate ocular vasoconstrictors due to adverse reactions, continued treatment with these may be stopped; and application of steroid eye drops for 3 days from the day of infusion (dexamethasone 0.1% eye drops or equivalent, 1 drop in each eye 3 times daily for 3 days [first drop to be given before start of tisotumab vedotin administration], otherwise to be used in accordance to product prescribing information). The ocular treatment guidelines are shown in Table 11.

TABLE 11
Ocular treatment guidelines

| Ocular symptom | Treatment guideline |
|---|---|
| (CTCAE grading) | (The length of treatment is decided by the local ophthalmologist) |
| Conjunctivitis grade 1 | The local ophthalmologist prescribes frequent dosing of |
| | preservative-free topical steroid drops. |
| Conjunctivitis grade 2 | The local ophthalmologist prescribes frequent dosing (every |
| | second hour) of preservative-free topical steroid drops in |
| | conjunction with preservative free antibiotic prophylaxis |
| | such as chloramphenicol. |
| Conjunctivitis grade 3 | The local ophthalmologist prescribes frequent dosing (every |
| | second hour) of preservative-free topical steroid drops in |
| | conjunction with preservative free antibiotic prophylaxis |
| | such as chloramphenicol. |
| Keratitis grade 1 | The local ophthalmologist prescribes frequent dosing of |
| | preservative-free topical steroid drops. |
| Keratitis grade 2 | The local ophthalmologist prescribes frequent dosing (every |
| | second hour) of preservative-free topical steroid drops in |
| | conjunction with preservative free antibiotic prophylaxis |
| | such as chloramphenicol. |
| Conjunctival | The local ophthalmologist prescribes frequent dosing (every |
| ulceration: Any grade | second hour) of preservative-free topical steroid drops in |
| | conjunction with preservative free antibiotic prophylaxis |
| | such as chloramphenicol. |

For AEs of peripheral neuropathy (including neuropathy peripheral; peripheral sensory neuropathy; peripheral motor neuropathy; polyneuropathy): Peripheral neuropathy is a well-known adverse reaction to treatment with chemotherapeutics (including cisplatin and taxanes) as well as MMAE-based ADCs and is frequently reported in relation to treatment with tisotumab vedotin. The majority of the reported cases are grade 1-2; however peripheral neuropathy is the leading cause of permanently discontinuation of tisotumab vedotin treatment. A mitigation plan, including dose reduction (see Table 9) and dose delay (i.e., hold dosing until event has improved to ≤grade 1), is in place to prevent onset of peripheral neuropathy as well as deterioration of pre-existing conditions. For AEs of bleeding: Bleeding events are considered of special interest due to the mode of action of tisotumab vedotin. In line with preclinical findings, no major impact on activated partial thromboplastin time (aPTT) or prothrombin time (PT) has until now been found for tisotumab vedotin treated patients. Epistaxis is the most common reported AE, however, nearly all of the cases are grade 1. Excluding epistaxis, no causal relation has been established for the majority of the reported bleeding events and treatment with tisotumab vedotin.

What is claimed is:

1. A method of treating cervical cancer in a subject, the method comprising administering to the subject an antibody-drug conjugate that binds to tissue factor (TF), wherein the antibody-drug conjugate comprises an anti-TF antibody or an antigen-binding fragment thereof conjugated to a monomethyl auristatin or a functional analog thereof or a functional derivative thereof, and wherein the antibody-drug conjugate is administered at a dose ranging from about 1.5 mg/kg to about 2.1 mg/kg.

2. The method of claim 1, wherein the dose is about 2.0 mg/kg.

3. The method of claim 1 or claim 2, wherein the antibody-drug conjugate is administered once about every 1 week, 2 weeks, 3 weeks or 4 weeks.

4. The method of any one of claims 1-3, wherein the antibody-drug conjugate is administered once about every 3 weeks.

5. The method of any one of claims 1-4, wherein the subject has been previously treated with one or more therapeutic agents and did not respond to the treatment, wherein the one or more therapeutic agents is not the antibody-drug conjugate.

6. The method of any one of claims 1-4, wherein the subject has been previously treated with one or more therapeutic agents and relapsed after the treatment, wherein the one or more therapeutic agents is not the antibody-drug conjugate.

7. The method of any one of claims 1-4, wherein the subject has been previously treated with one or more therapeutic agents and has experienced disease progression during treatment the, wherein the one or more therapeutic agents is not the antibody-drug conjugate.

8. The method of any one of claims 5-7, wherein the one or more therapeutic agents is a platinum-based therapeutic agent.

9. The method of any one of claims 5-7, wherein the one or more therapeutic agents is selected from the group consisting of: paclitaxel, cisplatin, carboplatin, topotecan, gemcitabine, fluorouracil, ixabepilone, imatinib mesylate, docetaxel, gefitinib, paclitaxel, pemetrexed, vinorelbine, doxil, cetuximab, pembrolizumab, nivolumab and bevacizumab.

10. The method of any one of claims 1-9, wherein the subject has experienced disease progression during or after treatment with:
a) paclitaxel and cisplatin,
b) paclitaxel and carboplatin, or
c) paclitaxel and topotecan.

11. The method of any one of claims 1-10, wherein the subject has received treatment with bevacizumab.

12. The method of any one of claims 1-10, wherein the subject is ineligible for treatment with bevacizumab.

13. The method of any one of claims 1-12, wherein the subject is not a candidate for curative therapy.

14. The method of claim 13, wherein the curative therapy comprises radiotherapy and/or exenterative surgery.

15. The method of any one of claims 1-14, wherein the subject did not respond to treatment with no more than two prior systemic treatment regimens.

16. The method of any one of claims 1-14, wherein the subject relapsed after treatment with no more than two prior systemic treatment regimens.

17. The method of any one of claims 1-16, wherein the cervical cancer is an adenocarcinoma, an adenosquamous carcinoma or a squamous cell carcinoma.

18. The method of any one of claims 1-17, wherein the cervical cancer is an advanced stage cervical cancer, such as a stage 3 or stage 4 cervical cancer, such as metastatic cervical cancer.

19. The method of any one of claims 1-18, wherein the cervical cancer is recurrent cervical cancer.

20. The method of any one of claims 1-19, wherein the monomethyl auristatin is monomethyl auristatin E (MMAE).

21. The method of any one of claims 1-20, wherein the anti-TF antibody or antigen-binding fragment thereof of the antibody-drug conjugate is a monoclonal antibody or a monoclonal antigen-binding fragment thereof.

22. The method of any one of claims 1-21, wherein the anti-TF antibody or antigen-binding fragment thereof of the antibody-drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and
wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6, wherein the CDRs of the anti-TF antibody or antigen-binding fragment thereof of the antibody-drug conjugate are defined by the IMGT numbering scheme.

23. The method of any one of claims 1-22, wherein the anti-TF antibody or antigen-binding fragment thereof of the antibody-drug conjugate comprises a heavy chain variable region comprising an amino acid sequence at least 85% identical to the amino acid sequence of SEQ ID NO:7 and a light chain variable region comprising an amino acid sequence at least 85% identical to the amino acid sequence of SEQ ID NO:8.

24. The method of any one of claims 1-23, wherein the anti-TF antibody or antigen-binding fragment thereof of the antibody-drug conjugate comprises a heavy chain variable region comprising the amino acid sequence of SEQ ID NO:7 and a light chain variable region comprising the amino acid sequence of SEQ ID NO:8.

25. The method of any one of claims 1-24, wherein the anti-TF antibody of the antibody-drug conjugate is tisotumab.

26. The method of any one of claims 1-25, wherein the antibody-drug conjugate further comprises a linker between the anti-TF antibody or antigen-binding fragment thereof and the monomethyl auristatin.

27. The method of claim 26, wherein the linker is a cleavable peptide linker.

28. The method of claim 27, wherein the cleavable peptide linker has a formula: -MC-vc-PAB-, wherein:
a) MC is: 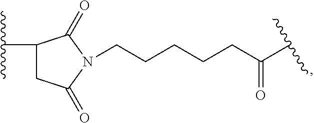 [SMILES: *C(=O)CCCCCN1C(=O)CC(*)C1=O]
b) vc is the dipeptide valine-citrulline, and
c) PAB is:

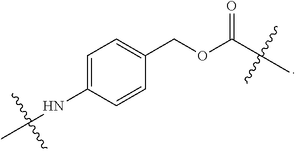 [SMILES: *Nc1ccc(COC(*)=O)cc1]

29. The method of any one of claims 26-28, wherein the linker is attached to sulphydryl residues of the anti-TF antibody obtained by partial reduction or full reduction of the anti-TF antibody or antigen-binding fragment thereof.

30. The method of claim 29, wherein the linker is attached to MMAE, wherein the antibody-drug conjugate has the following-structure: 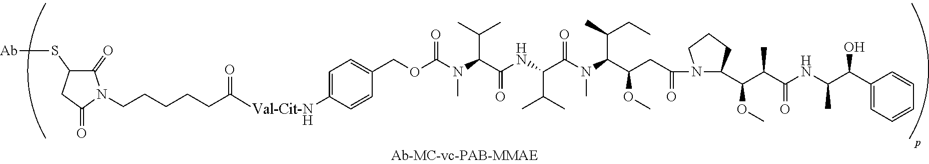
wherein p denotes a number from 1 to 8, S represents a sulphydryl residue of the anti-TF antibody, and Ab designates the anti-TF antibody or antigen-binding fragment thereof.

31. The method of claim 30, wherein the average value of p in a population of the antibody-drug conjugates is about 4.

32. The method of any one of claims 1-31, wherein the antibody-drug conjugate is tisotumab vedotin.

33. The method of any one of claims 1-32, wherein the route of administration for the antibody-drug conjugate is intravenous.

34. The method of any one of claims 1-33, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cervical cancer cells express TF.

35. The method of any one of claims 1-34, wherein one or more therapeutic effects in the subject is improved after administration of the antibody-drug conjugate relative to a baseline.

36. The method of claim 35, wherein the one or more therapeutic effects is selected from the group consisting of: size of a tumor derived from the cervical cancer, objective response rate, duration of response, time to response, progression free survival, and overall survival.

37. The method of any one of claims 1-36, wherein the size of a tumor derived from the cervical cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cervical cancer before administration of the antibody-drug conjugate.

38. The method of any one of claims 1-37, wherein the objective response rate is at least about 20%, at least about 25%, at least about 30%, at least about 35%, at least about 40%, at least about 45%, at least about 50%, at least about 60%, at least about 70%, or at least about 80%.

39. The method of any one of claims 1-38, wherein the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

40. The method of any one of claims 1-39, wherein the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

41. The method of any one of claims 1-40, wherein the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the antibody-drug conjugate.

42. The method of any one of claims 1-41, wherein the subject has one or more adverse events and is further administered an additional therapeutic agent to eliminate or reduce the severity of the one or more adverse events.

43. The method of any one of claims 1-41, wherein the subject is at risk of developing one or more adverse events and is further administered an additional therapeutic agent to prevent or reduce the severity of the one or more adverse events.

44. The method of claim 42 or claim 43, wherein the one or more adverse events is anemia, abdominal pain, hypokalemia, hyponatremia, epistaxis, fatigue, nausea, alopecia, conjunctivitis, constipation, decreased appetite, diarrhea, vomiting, peripheral neuropathy, or general physical health deterioration.

45. The method of claim 42 or claim 43, wherein the one or more adverse events is a grade 3 or greater adverse event.

46. The method of claim 42 or claim 43, wherein the one or more adverse events is a serious adverse event.

47. The method of claim 42 or claim 43, wherein the one or more adverse events is conjunctivitis and/or keratitis and the additional agent is a preservative-free lubricating eye drop, an ocular vasoconstrictor and/or a steroid eye drop.

48. The method of any one of claims 1-47, wherein the antibody-drug conjugate is administered as a monotherapy.

49. The method of any one of claims 1-48, wherein the subject is a human.

50. The method of any one of claims 1-49, wherein the antibody-drug conjugate is in a pharmaceutical composition comprising the antibody-drug conjugate and a pharmaceutical acceptable carrier.


SEQUENCE LISTING (16 sequences)

SEQ ID NO: 1 - protein, 8 aa, Artificial Sequence
  Synthetic Construct
GFTFSNYA

SEQ ID NO: 2 - protein, 8 aa, Artificial Sequence
  Synthetic Construct
ISGSGDYT

SEQ ID NO: 3 - protein, 11 aa, Artificial Sequence
  Synthetic Construct
ARSPWGYYLDS

SEQ ID NO: 4 - protein, 6 aa, Artificial Sequence
  Synthetic Construct
QGISSR

SEQ ID NO: 5 - protein, 3 aa, Artificial Sequence
  Synthetic Construct
AAS

SEQ ID NO: 6 - protein, 9 aa, Artificial Sequence
  Synthetic Construct
QQYNSYPYT

SEQ ID NO: 7 - protein, 118 aa, Artificial Sequence
  Synthetic Construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 8 - protein, 107 aa, Artificial Sequence
  Synthetic Construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 9 - protein, 25 aa, Artificial Sequence
  Synthetic Construct
EVQLLESGGGLVQPGGSLRLSCAAS

SEQ ID NO: 10 - protein, 17 aa, Artificial Sequence
  Synthetic Construct
MSWVRQAPGKGLEWVSS

SEQ ID NO: 11 - protein, 38 aa, Artificial Sequence
  Synthetic Construct
YYTDSVKGRFTISRDNSKNTLYLQMNSLRAEDTAVYYC

SEQ ID NO: 12 - protein, 11 aa, Artificial Sequence
  Synthetic Construct
WGQGTLVTVSS

SEQ ID NO: 13 - protein, 26 aa, Artificial Sequence
  Synthetic Construct
DIQMTQSPPSLSASAGDRVTITCRAS

SEQ ID NO: 14 - protein, 17 aa, Artificial Sequence
  Synthetic Construct
LAWYQQKPEKAPKSLIY

SEQ ID NO: 15 - protein, 36 aa, Artificial Sequence
  Synthetic Construct
SLQSGVPSRFSGSGSGTDFTLTISSLQPEDFATYYC

SEQ ID NO: 16 - protein, 10 aa, Artificial Sequence
  Synthetic Construct
FGQGTKLEIK